SEQUENCE LISTING (30 sequences)

SEQ ID NO: 1 - DNA, 21 bp, Artificial Sequence
  Synthetic oligonucleotide
gacggaggctaagcgtcgcaa

SEQ ID NO: 2 - DNA, 23 bp, Artificial Sequence
  Synthetic oligonucleotide
ggtgcctgataacctgagtatgg

SEQ ID NO: 3 - DNA, 24 bp, Artificial Sequence
  Synthetic oligonucleotide
gcataagcttccttcgtccagtgg

SEQ ID NO: 4 - DNA, 23 bp, Artificial Sequence
  Synthetic oligonucleotide
gcaggagaccttgtccgctgcgg

SEQ ID NO: 5 - DNA, 24 bp, Artificial Sequence
  Synthetic oligonucleotide
gatccgtggcccatcatgtcttgg

SEQ ID NO: 6 - DNA, 23 bp, Artificial Sequence
  Synthetic oligonucleotide
gttgtgctgtgtatggacgtggg

SEQ ID NO: 7 - DNA, 24 bp, Artificial Sequence
  Synthetic oligonucleotide
gccaaggctattcagtgtgcgagg

SEQ ID NO: 8 - DNA, 24 bp, Artificial Sequence
  Synthetic oligonucleotide
gtgatagagctacagaacgaaagg

SEQ ID NO: 9 - DNA, 24 bp, Artificial Sequence
  Synthetic oligonucleotide
gaagacctgcaaccgtgcccgggg

SEQ ID NO: 10 - DNA, 19 bp, Artificial Sequence
  Synthetic oligonucleotide
agctccaggcccggattcg

SEQ ID NO: 11 - DNA, 20 bp, Artificial Sequence
  Synthetic oligonucleotide
tgcccgttctccagaagctc

SEQ ID NO: 12 - DNA, 18 bp, Artificial Sequence
  Synthetic oligonucleotide
ccctactgtatcctcatg

SEQ ID NO: 13 - DNA, 20 bp, Artificial Sequence
  Synthetic oligonucleotide
cttacctcctcttcaatagc

SEQ ID NO: 14 - DNA, 20 bp, Artificial Sequence
  Synthetic oligonucleotide
ggggcatttccgggtccggg

SEQ ID NO: 15 - DNA, 20 bp, Artificial Sequence
  Synthetic oligonucleotide
tgccctgccccccactctgc

SEQ ID NO: 16 - DNA, 21 bp, Artificial Sequence
  Synthetic oligonucleotide
aacagcctcaagatcatcagc

SEQ ID NO: 17 - DNA, 21 bp, Artificial Sequence
  Synthetic oligonucleotide
caccaccttcttgatgtcatc

SEQ ID NO: 18 - DNA, 21 bp, Artificial Sequence
  Synthetic oligonucleotide
acgccgcattgaccatctatg

SEQ ID NO: 19 - DNA, 22 bp, Artificial Sequence
  Synthetic oligonucleotide
cggaggtaacctgtaagtctgt

SEQ ID NO: 20 - DNA, 24 bp, Artificial Sequence
  Synthetic oligonucleotide
gttggatacaggccagatttgttg

SEQ ID NO: 21 - DNA, 23 bp, Artificial Sequence
  Synthetic oligonucleotide
gagggtaggctggcctataggct

SEQ ID NO: 22 - DNA, 24 bp, Artificial Sequence
  Synthetic oligonucleotide
gcactgggtggaatgagactattg

SEQ ID NO: 23 - DNA, 24 bp, Artificial Sequence
  Synthetic oligonucleotide
ttctgaggcatcaactgacaggtc

SEQ ID NO: 24 - protein, 13 aa, Artificial Sequence
  IRF3 phosphorylation domain on serine 386
RVGGASSLENTVD

SEQ ID NO: 25 - protein, 15 aa, Artificial Sequence
  HSPA8 phosphorylation domain on serine 638
PSGGASSGPTIEEVD

SEQ ID NO: 26 - protein, 16 aa, Artificial Sequence
  IRF3 phosphorylation domain on serine 386
MARVGGASSLENTVDL

SEQ ID NO: 27 - protein, 16 aa, Artificial Sequence
  IRF3 phosphorylation domain on serine 386
IARVGGASSLENTVDL

SEQ ID NO: 28 - protein, 16 aa, Artificial Sequence
  IRF3 phosphorylation domain on serine 386
LARVGGASSLENTVEL

SEQ ID NO: 29 - protein, 15 aa, Artificial Sequence
  IRF3 phosphorylation domain on serine 386
MAREGGASSLRTVDL

SEQ ID NO: 30 - protein, 15 aa, Artificial Sequence
  IRF3 phosphorylation domain on serine 386
MAREGGASSLKTVDL

(19) United States
(12) Patent Application Publication
(10) Pub. No.: US 2022/0143035 A1
(43) Pub. Date: May 12, 2022

(54) METHODS FOR TREATING AUTOIMMUNE OR AUTOINFLAMMATORY DISEASE

(71) Applicant: UNIVERSITY OF WASHINGTON, Seattle, WA (US)

(72) Inventors: Daniel Brewster STETSON, Seattle, WA (US); Katelyn BURLEIGH, Seattle, WA (US); Joanna MALTBAEK, Seattle, WA (US)

(21) Appl. No.: 17/435,194

(22) PCT Filed: Mar. 3, 2020
(86) PCT No.: PCT/US20/20747

§ 371 Date: Aug. 31, 2021

Related U.S. Application Data
(60) Provisional application No. 62/813,482, filed on Mar. 4, 2019.
(60) Provisional application No. 62/964,865, filed on Jan. 23, 2020.

(51) Int. Cl. A61K 31/5377; A61K 31/7105; A61K 31/711; A61K 39/395; A61K 31/5375; A61K 31/519; A61K 31/4184; A61K 31/341; A61K 31/404; A61P 37/06; G01N 33/564

(52) U.S. Cl. CPC A61K 31/5377; A61K 31/7105; A61K 31/711; A61K 39/3955; A61K 31/5375; G01N 2800/52; A61K 31/4184; A61K 31/341; A61K 31/404; A61P 37/06; G01N 33/564; A61K 31/519

(57) ABSTRACT

The disclosure provides method and compositions for treating of an autoimmune disease or an autoinflammatory disease, including administering to a subject in need thereof an amount effective of a DNA-dependent protein kinase (DNA-PK) inhibitor and/or an inhibitor of HSPA8/HSC70.

### CROSS REFERENCE

This application claims priority to U.S. Provisional Patent Application Ser. Nos. 62/813,482 filed Mar. 4, 2019 and 62/964,865 filed Jan. 23, 2020, each incorporated by reference herein in their entirety.

### FEDERAL FUNDING STATEMENT

This invention was made with government support under Grant No. R21 AI130940, awarded by the National Institutes of Health (NIH). The government has certain rights in the invention.

### REFERENCE TO SEQUENCE LISTING

This application contains a Sequence Listing submitted as an electronic text file named “19-334-PCT_SequenceListing_ST25.txt”, having a size in bytes of 7 kb, and created on Feb. 21, 2020. The information contained in this electronic file is hereby incorporated by reference in its entirety pursuant to 37 CFR § 1.52(e)(5).

### BACKGROUND

The Cyclic GMP-AMP synthase (cGAS)-Stimulator of interferon genes (STING) DNA sensing pathway has emerged as a key component of the innate immune response that is important for antiviral immunity, contributes to specific autoimmune diseases, and mediates important aspects of antitumor immunity. cGAS binds to double-stranded DNA and catalyzes the formation of cyclic GMP-AMP (cGAMP), a diffusible cyclic dinucleotide that activates the endoplasmic adaptor protein STING. Activated STING then serves as a platform for the inducible recruitment of the TBK1 kinase, which phosphorylates and activates the transcription factor IRF3, leading to the induction of the type I interferon mediated antiviral response. It is unclear whether STING-independent DNA sensing pathways are present in human cells.

### SUMMARY

In a first aspect, the disclosure provides methods for treating of an autoimmune disease or an autoinflammatory disease, comprising administering to a subject in need thereof an amount effective of a DNA-dependent protein kinase (DNA-PK) inhibitor and/or an inhibitor of HSPA8/HSC70, to treat the autoimmune disorder or the auto-inflammatory disorder. In one embodiment, the DNA-PK inhibitor and/or the HSPA8/HSC70 inhibitor are not inhibitors expressed by non-recombinant viruses. In another embodiment, the method comprises administering the DNA-PK inhibitor to the subject, wherein the DNA-PK inhibitor comprises one or more of small molecule inhibitors of activity (such as kinase activity), antisense oligonucleotides directed against the DNA-PK DNA or mRNA; small interfering RNAs (siRNAs), short hairpin RNAs (shRNAs), microRNAs (miRNA) or small internally segmented interfering RNAs (sisiRNA) directed against the DNA-PK protein, DNA, or mRNA; DNA-PK antibodies, and aptamers that bind to DNA-PK. In a further embodiment, the DNA-PK inhibitor is a small molecule inhibitor, including to but not limited to small molecule inhibitors disclosed herein such as NU-7441, M3814, Compound II (2-(Morpholin-4-yl)-benzo[h]chromen-4-one), or Compound III (1-(2-hydroxy-4-morpholinophenyl)ethan-1-one), or pharmaceutically acceptable salts, esters, or prodrugs thereof, or pharmaceutically acceptable salts, esters, or prodrugs thereof.

In one embodiment, the method comprises administering the HSPA8/HSC70 inhibitor to the subject. In a further embodiment, the HSPA8/HSC70 inhibitor comprises a small molecule inhibitor of activity, antisense oligonucleotides directed against the HSPA8/HSC70 DNA or mRNA; small interfering RNAs (siRNAs), short hairpin RNAs (shRNAs), microRNAs (miRNA) or small internally segmented interfering RNAs (sisiRNA) directed against the HSPA8/HSC70 protein, DNA, or mRNA; HSPA8/HSC70 antibodies, aptamers that bind to HSPA8/HSC70, and any other chemical or biological compound that can interfere with HSPA8/HSC70 expression, activity, and/or stability.

In another embodiment, the method further comprises administering an inhibitor of Cyclic GMP-AMP synthase (cGAS) expression, activity, and/or stability, and/or an inhibitor of Stimulator of interferon genes (STING), also known as transmembrane protein 173 (TMEM173)) expression, activity, and/or stability. In one such embodiment, the cGAS and/or STING inhibitor may include, but it not limited to, small molecule inhibitors, antisense oligonucleotides directed against the cGAS or STING DNA or mRNA; small interfering RNAs (siRNAs), short hairpin RNAs (shRNAs), microRNAs (miRNA) or small internally segmented interfering RNAs (sisiRNA) directed against the cGAS or STING protein, DNA, or mRNA; cGAS or STING antibodies, aptamers that bind to cGAS or STING, any other chemical or biological compound that can interfere with cGAS or STING expression, activity, and/or stability.

In one embodiment, the subject has an autoimmune disease. In a further embodiment, the autoimmune disease comprises one or more of Systemic lupus erythematosus (SLE), Discoid lupus, Cutaneous lupus, Sjogrens syndrome, Aicardi-Goutieres syndrome (AGS), pemphigoid (any type), Crohn's disease, endometriosis, fibromyalgia, glomerulonephritis, juvenile arthritis, type 1 diabetes, multiple sclerosis, psoriasis, rheumatoid arthritis, sarcoidosis, scleroderma, and ulcerative colitis. In another embodiment, the subject has an autoinflammatory disease.

In another aspect, the disclosure provides methods for monitoring therapy of a subject being treated for an autoimmune disease and/or an autoinflammatory disease, comprising

(a) determining a baseline level of HSPA8/HSC70 phosphorylation in a biological sample from the subject; and

(b) determining level of HSPA8/HSC70 phosphorylation in a biological sample from the subject 1 or more (2, 3, 4, 5, 6, or more times) after treatment for the autoimmune disease and/or an autoinflammatory disease,

wherein a decrease in HSPA8/HSC70 phosphorylation in the biological sample from the subject after treatment indicates efficacy of the therapy, and wherein an increase in HSPA8/HSC70 phosphorylation in the biological sample from the subject after treatment indicates that the therapy was ineffective. In one embodiment, determining the level of HSPA8/HSC70 phosphorylation comprises determining phosphorylation of serine 638 of human HSPA8/HSC70.

In a further aspect the disclosure provides methods for identifying compounds to treat autoimmune disease and/or autoinflammatory diseases, comprising identifying compounds that inhibit DNA-PK and/or HSPA8/HSC70 expression, activity, and/or stability. In one embodiment, the method comprises identifying compounds that inhibit DNA-PK phosphorylation of HSPA8/HSC70. In another embodiment, the method comprises identifying compounds that inhibit DNA-PK phosphorylation of serine 638 of HSPA8/HSC70.

In another aspect, the disclosure provides pharmaceutical compositions, comprising:

(a) a DNA-PK inhibitor and/or an inhibitor of HSPA8/HSC70; and

(b) an inhibitor of cGAS expression, activity, and/or stability, and/or an inhibitor of STING expression, activity, and/or stability. In one embodiment, the DNA-PK inhibitor and/or an inhibitor of HSPA8/HSC70 comprises a DNA-PK inhibitor. In another embodiment, the DNA-PK inhibitor comprises one or more of small molecule inhibitors of activity (such as kinase activity), antisense oligonucleotides directed against the DNA-PK DNA or mRNA; small interfering RNAs (siRNAs), short hairpin RNAs (shRNAs), microRNAs (miRNA) or small internally segmented interfering RNAs (sisiRNA) directed against the DNA-PK protein, DNA, or mRNA; DNA-PK antibodies, and aptamers that bind to DNA-PK. In a further embodiment, the DNA-PK inhibitor is a small molecule inhibitor, including but not limited to the DNA-PK inhibitors disclosed herein such as NU-7441, M3814, Compound II (2-(Morpholin-4-yl)-benzo[h]chromen-4-one), or Compound III (1-(2-hydroxy-4-morpholinophenyl)ethan-1-one), or pharmaceutically acceptable salts, esters, or prodrugs thereof, or pharmaceutically acceptable salts, esters, or prodrugs thereof. In one embodiment, the pharmaceutical composition comprises an HSPA8/HSC70 inhibitor. In a further embodiment, the HSPA8/HSC70 inhibitor comprises a small molecule inhibitor of activity, antisense oligonucleotides directed against the HSPA8/HSC70 DNA or mRNA; small interfering RNAs (siRNAs), short hairpin RNAs (shRNAs), microRNAs (miRNA) or small internally segmented interfering RNAs (sisiRNA) directed against the HSPA8/HSC70 protein, DNA, or mRNA; HSPA8/HSC70 antibodies, aptamers that bind to HSPA8/HSC70, and any other chemical or biological compound that can interfere with HSPA8/HSC70 expression, activity, and/or stability. In another embodiment, the pharmaceutical composition comprises a cGAS inhibitor. In one such embodiment, the cGAS inhibitor comprises a small molecule cGAs inhibitor, antisense oligonucleotides directed against the cGAS DNA or mRNA; small interfering RNAs (siRNAs), short hairpin RNAs (shRNAs), microRNAs (miRNA) or small internally segmented interfering RNAs (sisiRNA) directed against the cGAS protein, DNA, or mRNA; cGAS antibodies, aptamers that bind to cGAS, and any other chemical or biological compound that can interfere with cGAS expression, activity, and/or stability.

In a further embodiment, the pharmaceutical composition comprises a STING inhibitor. In one such embodiment, the STING inhibitor comprises a small molecule STING inhibitor, antisense oligonucleotides directed against the STING DNA or mRNA; small interfering RNAs (siRNAs), short hairpin RNAs (shRNAs), microRNAs (miRNA) or small internally segmented interfering RNAs (sisiRNA) directed against the STING protein, DNA, or mRNA; STING antibodies, aptamers that bind to STING, and any other chemical or biological compound that can interfere with STING expression, activity, and/or stability. In a further embodiment, the DNA-PK inhibitor, the HSPA8/HSC70 inhibitor, the cGAS inhibitor, and the STING inhibitor are not inhibitors expressed by non-recombinant viruses.

### DESCRIPTION OF THE FIGURES

FIG. 1A-D: Human Adenovirus 5 E1A blocks two DNA sensing pathways. (A) HEK 293 cells were transduced with lentiCRISPR encoding H1 control or E1A-specific gRNAs, selected for three days, and then stimulated with CT DNA or RIG-I ligand for 24 hours, followed by measuring type I IFN activity in culture supernatants. *:p<0.01. n=3 independent treatments per condition. Error bars represent Standard Deviation (SD). (B) HEK 293 cells, treated with the indicated ligands, were lysed at the indicated times post transfection, followed by western blot analysis for phosphorylated TBK1 and IRF3. (C) Clonal lines of H1 control-targeted, STING KO, and TBK1 KO HEK 293 cells were treated with the indicated ligands for 3 hours, followed by western blot for the indicated phosphorylation sites on IRF3. (D) Clonal lines of H1 control-targeted and STING KO HEK 293 cells were transduced with lentiCRISPR as described in (A), then stimulated with the indicated ligands for 8 hours followed by western blot analysis. Data are representative of three independent experiments per panel.

FIG. 2A-F: A STING-independent DNA sensing pathway (SIDSP) in human cells. (A) Primary mouse embryonic fibroblasts were treated with Lipofectamine alone (Lipo) or the indicated ligands for four hours before harvest and quantitative RT-PCR (RT-qPCR) analysis of Ifnb mRNA expression. n=3 independent treatments per condition. (B) PMA-differentiated human U937 monocytes or two clonal lines of STING KO U937 cells were treated with the indicated ligands for 16 hours before harvest and RT-qPCR analysis of IFNB1 mRNA expression. n=3 independent treatments per condition. (C) PMA-differentiated WT U937 cells and STING KO U937 cells were treated with the indicated ligands for the indicated times before harvest and RT-qPCR analysis of IFNB1 mRNA expression. n.s.: not significant. n=3 independent treatments per condition. (D) Tert-HFF cells were transduced with lentiCRISPR encoding either H1 control non-targeting gRNA or STING targeting gRNA, followed by selection for three days in puromycin. STING expression was assessed by western blot. (E) Tert-HFFs from (D) were treated with the indicated ligands for 16 hours prior to harvest and RT-qPCR for IFNB1. Multiple t-tests with significance determined by Holm-Sidak method were used to compare cell lines for each stimulation, n=3 independent treatments per condition. (F) Clonal lines of PMA-differentiated H1 control-targeted THP1 cells and IRF3/IRF7 DKO THP1 cells were treated with the indicated ligands for 16 hours before harvest and RT-qPCR analysis of IFNB1 mRNA expression. n=3 independent treatments per condition. Error bars represent SD. Data are representative of 3 independent experiments per panel.

FIG. 3A-E: The SIDSP is activated by DNA ends. (A) PMA-differentiated clonal lines of H1 control-targeted and STING KO U937 cells were treated with the indicated ligands for 16 hours before harvest and RT-qPCR analysis of IFNB1 mRNA expression. n.s.: not significant; **:p<0.01. (B) CT DNA, supercoiled plasmid DNA, and sonicated plasmid DNA were run on a DNA agarose gel and visualized with SYBR-Safe. (C) Two clonal lines of PMA-differentiated STING KO U937 cells were treated with the indicated ligands for 16 hours, with IFNB1 mRNA expression in CT DNA-treated cells set at 100%. n=3 independent treatments per condition. (D) CT DNA, 100 base pair annealed DNA oligos (ISD100), supercoiled and sonicated plasmid DNAs were visualized on DNA-agarose gel. (E) STING KO HEK 293 cells were treated with the indicated ligands for three hours before harvesting lysates and evaluating IRF3 S386 phosphorylation by western blot. Error bars represent SD. Data are representative of 3 independent experiments per panel.

FIG. 4A-K: Human DNA-PK is essential for the SIDSP. (A) PMA-differentiated STING KO U937 cells were treated with CT DNA for 16 hours in the presence of DMSO control, increasing concentrations of Ku-60019 ATM inhibitor [0.125, 0.25, 0.5, 1 μM], or Nu-7441 DNA-PK inhibitor [0.25, 0.5, 1, 2 μM], followed by western blot analysis of γ-H2AX phosphorylation. (B) PMA-differentiated STING KO U937 cells were treated with CT DNA for 16 hours in the presence of inhibitors as described in (A), followed by RT-qPCR analysis of IFNB1 mRNA expression. n.s.: not significant; ***:p<0.001. n=3 independent treatments per condition. (C) PMA-differentiated STING KO U937 cells were treated with CT DNA or RIG-I ligand in the presence of DMSO or Nu-7441 for 16 hours, followed by RT-qPCR analysis of IFNB1 mRNA expression. n.s.: not significant; ***:p<0.001. n=3 independent treatments per condition. (D) Western blot analysis of DNA-PK and STING in clonal lines of H1 control, STING KO and STING/DNA-PK DKO U937 cells. (E) PMA-differentiated STING KO and STING/DNA-PK DKO U937 cells were treated with CT DNA for 16 hours in DMSO or 2 μM Nu-7441, followed by RT-qPCR analysis of IFNB1 mRNA expression, normalized to Lipo control-treated cells. **:p<0.01. n=3 independent treatments per condition. (F) Primary human foreskin fibroblasts (HFF) and primary human hepatocytes from male (♂) and female (♀) donors were assessed for cGAS, STING, and DNA-PK proteins by fractionation into cytosol (C) and nuclear (N) extracts, followed by western blot. The nuclear extract was treated with salt-active nuclease to remove genomic DNA. (G) The indicated cell lines were stimulated with cGAMP for 16 hours before harvest and RT-qPCR analysis of IFNB1 mRNA expression. One-way ANOVA with Holm Sidak's multiple comparisons test was used to compare IFNB1 in the HFFs versus the hepatocytes. ****:p<0.0001. n=3 independent treatments per condition. (H) The indicated cell lines were stimulated for 4 hours before measurement of cGAMP by ELISA. n.s.: not significant; *:p<0.05. n=3 independent treatments per condition. (I) The indicated cell lines were treated with CT DNA for 16 hours in the presence of DMSO or 2 μM Nu-7441 before harvest and RT-qPCR analysis of IFNB1 mRNA expression. Multiple t-tests with significance determined by the Holm-Sidak method were used to compare IFNB1 in DMSO versus Nu7441 treated cells. *:p<0.05, ****:P<.0001. n=3 independent treatments per condition. (J) STING KO HEK 293 cells were transduced with lentiCRISPR encoding gRNAs specific for the indicated targets, selected for three days in puromycin, and then harvested for western blot analysis of the indicated proteins. (K) STING KO HEK 293 cells from (J) were stimulated with CT DNA for the indicated time points and then harvested for western blot analysis of IRF3 S386 phosphorylation. Error bars represent SD. Data are representative of 3 independent experiments per panel.

FIG. 5A-F: The DNA-PK SIDSP activates a broad gene expression program. (A) Heat map representation of log 2 Fold Change in gene expression for the 124 antiviral response genes with significant differential expression in one of the four comparisons. The key includes a histogram in cyan plotting the distribution of log 2 Fold Change values for all the included genes. Data represent the average of three independent treatments per condition. (B) Expression data for all interferon genes significantly induced in clonal lines of H1 control targeted or STING KO U937 cells, plotting the log 2 Fold Change at 8 and 16 hours post treatment. Data represent the average of three independent treatments per condition. (C) To measure the trajectories of global gene expression from 8 to 16 hours post CT DNA transfection, the fold change in gene expression at 16 hours was divided by the fold change for the same genes at 8 hours and plotted for all upregulated genes in STING KO (n=926) and H1 control lines (n=563). ****:p<0.0001, Mann-Whitney unpaired t-test. Data represent the average of three independent treatments per condition. (D) In H1 control U937 cells, the 16 hour CT DNA-activated log 2 Fold Change was plotted for DMSO control-treated cells on the x-axis and for Nu-7441-treated cells on the y-axis. Each dot represents a single gene that was differentially expressed at 16 hours in DMSO-treated cells (n=1024). The red line through the origin indicates the line of equivalence representing no effect of the drug treatment. A Wilcoxon matched pairs signed rank test was used to determine the p-value between DMSO control and Nu-7441-treated samples after DNA stimulation. Data represent the average of three independent treatments per condition. (E) In STING KO U937 cells, the effect of Nu-7441 on global gene expression for 1327 genes differentially expressed in DMSO control-treated cells, plotted as in (D). A Wilcoxon matched pairs signed rank test was used to determine the p value between DMSO control and Nu-7441-treated samples after DNA stimulation. Data represent the average of three independent treatments per condition. (F) In STING KO U937 cells, the log 2 Fold Change in DMSO control-treated cells was plotted on the y-axis for differentially expressed genes, and the effect of Nu-7441 on these same genes was plotted in log 10 format on the x-axis, n=1327. Data represent the average of three independent treatments per condition, each processed and sequenced independently.

FIG. 6A-M: HSPA8 is a downstream target of the DNA-PK-SIDSP. (A-D): The indicated human cells were treated with CT DNA or RIG-I ligand for the indicated times before harvest and western blot analysis of IRF3 S386 phosphorylation. Mystery Protein is indicated as MP on the blots. (E) Clonal lines of H1 non-targeting control, STING KO, and TBK1 KO HEK 293 cells were treated with the indicated ligands for 3 hours before harvest and western blot analysis of MP. (F) STING KO HEK 293 cells were treated with the DNA ligands described in FIG. 3D for 3 hours, followed by western blot analysis of MP. (G) Alignments of human IRF3 (SEQ ID NO:24) and HSPA8/HSC70 (SEQ ID NO:25). The red S indicates IRF3 S386 and HSPA8 S638. (H) HEK 293 cells were transfected with plasmids encoding the indicated human HA-HSPA8 constructs, then treated the next day with CT DNA for 3 hours before harvest, HA-immunoprecipitation, and western blot using the IRF3 pS386 antibody. (I) HEK 293 cells targeted for the indicated genes were treated with DNA and harvested for western blot analysis using the IRF3 pS386 antibody that detects HSPA8 pS638. (J) STING KO HEK 293 cells were transduced with lentiCRISPR targeting H1 control, DNA-PK, or ATM, selected for three days, and then harvested for western blot of the indicated proteins. (K) STING KO HEK 293 cells, transduced and selected as described in (J), were treated with CT DNA and then harvested for western blot analysis of IRF3 S386 and HSPA8 S638 phosphorylation. (L) STING KO HEK 293 cells were transfected with plasmid encoding the ICP0 protein of herpes simplex virus 1. 24 hours later, the cells were stimulated with CT DNA for 3 hours before harvest and western blot analysis of the indicated proteins. (M) STING KO HEK 293 cells were infected with increasing doses of either WT or ICP0-null mutant HSV1 (doses are MOI of 0, 0.1, 1, and 10) for 4 hours before 3 hour treatment with CT DNA and harvest for western blot. Data are representative of 3 independent experiments per panel.

FIG. 7A-E: HSPA8 phosphorylation delineates the antiviral modality of human DNA-PK. (A) The indicated human, primate, and mouse cell lines were stimulated with CT-DNA for 3 hours before harvest and western blot for the indicated proteins. (B) Primary human fibroblasts (HFF) and primary mouse embryonic fibroblasts from C57BL/6, CAST/Ei, PWK, and WSB mice were transfected with CT DNA for 6 hours before harvest and evaluation of the indicated proteins by western blot. (C) HEK 293 cells were transfected with either human HA-HSPA8 constructs or mouse HA-HSPA8 constructs, followed by CT DNA stimulation for 3 hours, HA immunoprecipitation, and western blot analysis of the indicated proteins. (D) Immortalized mouse Jackson fibroblasts were transfected and treated as indicated in (B). (E) HEK 293 cells were stimulated with CT DNA or supercoiled plasmid DNA, or treated with 30 Gray ionizing γ-irradiation, 50 μM Etoposide, or 500 nM Thapsigargin before harvest at the indicated time points and western blot analysis of IRF3 pS386, HSPA8 pS638, and γ-H2AX. Data are representative of 2-3 independent experiments per panel.

FIG. 8A-E: Normalized mRNA-Seq data comparing WT and STING KO U937 cells. (A) Boxplot depicting normalized read counts in log 2CPM format across all 36 samples. (B) A Metrics Dimensional Scaling (MDS) plot, color coded for the three biological replicates of each condition. (C) Normalized read counts in log 2CPM format, comparing Lipo-treated WT U937 cells to Lipo-treated STING KO U937 cells. (D) Normalized read counts in log 2CPM format, comparing Lipo-treated WT U937 cells pretreated with DMSO or 2 μM N-7441. (E) Normalized read counts in log 2CPM format, comparing Lipo-treated STING KO cells pretreated with DMSO or 2 μM N-7441.

FIG. 9A-D: Characterization of HSPA8 phosphorylation on serine 638. (A) HEK 293 cells were stimulated with CT DNA or RIG-I ligand for 3 hours followed by preparation of extracts that were either left untreated or treated with alkaline phosphatase prior to western blot analysis of IRF3 pS386 and MP. (B) Control and IRF3-CRISPR cells were transfected for three hours with CT-DNA, followed by preparation of cell lysates and immunoprecipitation using IRF3 pS386 antibody. Lysates before and after IP and the IP'd material were analyzed by western blot. (C) Alignments of HSPA8 and IRF3 amino acid sequences surrounding the phosphorylated serines (Homo sapiens HSPA8 (SEQ ID NO:25) and IRF3 (SEQ ID NO:26); Pan troglodytes HSPA8 (SEQ ID NO:25) and IRF3 (SEQ ID NO:26); Macaca mulatta HSPA8 (SEQ ID NO:25) and IRF3 (SEQ ID NO:27); Chlorocebus aethiops (SEQ ID NO:25) and IRF3 (SEQ ID NO:27); Aotus trivirgatus HSPA8 (SEQ ID NO:25) and IRF3 (SEQ ID NO:28); Saimiri boliviensis (SEQ ID NO:25) and IRF3 (SEQ ID NO:28); Mesocricetus auratus (SEQ ID NO:25) and IRF3 (SEQ ID NO:29); Rattus norvegicus HSPA8 (SEQ ID NO:25) and IRF3 (SEQ ID NO:30); and Mus musculus HSPA8 (SEQ ID NO:25) and IRF3 (SEQ ID NO:30)). (D) Cell lines from the indicated species were stimulated with CT DNA for 3 hours, followed by analysis of IRF3 pS386 and HSPA8 pS638 by western blot.

### DETAILED DESCRIPTION

As used herein, the singular forms “a”, “an” and “the” include plural referents unless the context clearly dictates otherwise.

All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application. The compositions and methods for their use can “comprise,” “consist essentially of,” or “consist of” any of the ingredients or steps disclosed throughout the specification.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In a first aspect, the invention provides methods for treating of an autoimmune disease or an autoinflammatory disease, comprising administering to a subject in need thereof an amount effective of a DNA-dependent protein kinase (DNA-PK) inhibitor and/or an inhibitor of HSPA8/HSC70, to treat the autoimmune disorder or the auto-inflammatory disorder. As disclosed herein, the inventors have identified DNA-PK as a potent, STING-independent DNA sensing pathway (SISDP) that is blocked by the E1A viral oncogene of human adenovirus 5 and the ICP0 product of herpes simplex virus 1. The inventors have further demonstrated that DNA-PK kinase activity drives a robust and broad antiviral response, that the heat shock protein HSPA8/HSC70 is a unique target of the DNA-PK SIDSP, and that detection of foreign DNA and DNA damage trigger distinct modalities of DNA-PK activity. The data demonstrate the utility of DNA-PK and HSPA8/HSC70inhibitors in autoimmune and autoinflammatory disorders, such as those mediated by interferon. In one embodiment, the DNA-PK inhibitor and/or the HSPA8/HSC70 inhibitor are not inhibitors expressed by non-recombinant viruses. In another embodiment, the DNA-PK inhibitor and/or the HSPA8/HSC70 inhibitor are not naturally occurring inhibitors.

DNA-PK is a DNA-activated serine/threonine protein kinase composed of a heterodimer of Ku proteins (Ku70/Ku80) and the catalytic subunit DNA-PKcs, is a critical component of the response to damage, and is present in a wide variety of species.

Any suitable inhibitor of DNA-PK expression and/or activity (such as kinase activity) may be used in the methods disclosed herein. In various embodiments, the inhibitor may comprise small molecule inhibitors of activity (such as kinase activity), antisense oligonucleotides directed against the DNA-PK DNA or mRNA; small interfering RNAs (siRNAs), short hairpin RNAs (shRNAs), microRNAs (miRNA) or small internally segmented interfering RNAs (sisiRNA) directed against the DNA-PK protein, DNA, or mRNA; DNA-PK antibodies, aptamers that bind to DNA-PK, and any other chemical or biological compound that can interfere with DNA-PK expression, activity (such as kinase activity), and/or stability. Based on the present disclosure in light of the level of skill in the art, those of skill in the art can readily identify other DNA-PK inhibitors. In one embodiment, the DNA-PK inhibitor is a small molecule inhibitor. In specific embodiments, the DNA-PK small molecule inhibitor comprises one or more of NU-7441, M3814, Compound II, or Compound III (all shown below), or pharmaceutically acceptable salts, esters, or prodrugs thereof.

### NU-7441 (8-(dibenzo[b,d]thiophen-4-yl)-2-morpholino-4H-chromen-4-one)

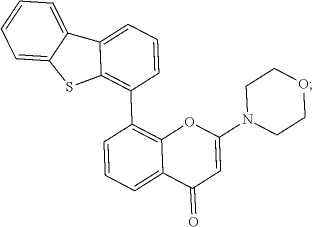 [SMILES: O=c1cc(N2CCOCC2)oc2c(-c3cccc4c3sc3ccccc34)cccc12]

### Nedisertib ((S)-[2-chloro-4-fluoro-5-(7-morpholin-4-ylquinazolin-4-yl)phenyl]-(6-methoxypyridazin-3-yl)methanol; M3814)

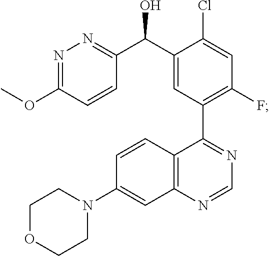 [SMILES: COc1ccc([C@@H](O)c2cc(-c3ncnc4cc(N5CCOCC5)ccc34)c(F)cc2Cl)nn1]

### Compound II (2-(Morpholin-4-yl)-benzo[h]chromen-4-one)

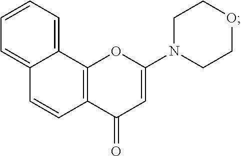 [SMILES: O=c1cc(N2CCOCC2)oc2c1ccc1ccccc12]

### Compound III (1-(2-hydroxy-4-morpholinophenyl)ethan-1-one)

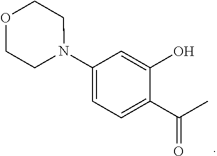 [SMILES: CC(=O)c1ccc(N2CCOCC2)cc1O]

In another embodiment, the method comprises administering the HSPA8/HSC70 inhibitor to the subject. Any suitable inhibitor of HSPA8/HSC70 expression and/or activity may be used in the methods disclosed herein. In various embodiments, the inhibitor may comprise small molecule inhibitor of activity, antisense oligonucleotides directed against the HSPA8/HSC70 DNA or mRNA; small interfering RNAs (siRNAs), short hairpin RNAs (shRNAs), microRNAs (miRNA) or small internally segmented interfering RNAs (sisiRNA) directed against the HSPA8/HSC70 protein, DNA, or mRNA; HSPA8/HSC70 antibodies, aptamers that bind to HSPA8/HSC70, and any other chemical or biological compound that can interfere with HSPA8/HSC70 expression, activity, and/or stability.

In another embodiment, the method further comprises administering an inhibitor of Cyclic GMP-AMP synthase (cGAS) expression, activity, and/or stability, and/or an inhibitor of Stimulator of interferon genes (STING), also known as transmembrane protein 173 (TMEM173)) expression, activity, and/or stability. This embodiment provides combined therapies targeting separate immune system activation pathways, and thus provided added therapeutic benefit. Non-limiting, exemplary such inhibitors can include small molecule inhibitors, antisense oligonucleotides directed against the cGAS or STING DNA or mRNA; small interfering RNAs (siRNAs), short hairpin RNAs (shRNAs), microRNAs (miRNA) or small internally segmented interfering RNAs (sisiRNA) directed against the cGAS or STING protein, DNA, or mRNA; cGAS or STING antibodies, aptamers that bind to cGAS or STING, and any other chemical or biological compound that can interfere with cGAS or STING expression, activity, and/or stability. In some embodiments, the cGAS inhibitor may comprise any cGAS inhibitors, such as PF-06928215, disclosed in PLos One 2017 Sep. 21; 12(9):e0184843. doi: 10.1371/journal. pone.0184843. eCollection 2017 and/or RU.521 (Nat Commun. 2017 Sep. 29; 8(1):750. doi: 10.1038/s41467-017-00833-9), or pharmaceutically acceptable salts, esters, or prodrugs thereof. In another embodiment, the STING inhibitor may comprise one or more STING inhibitors disclosed in Nature. 2018 July; 559(7713):269-273. doi: 10.1038/s41586-018-0287-8. Epub 2018 Jul. 4.

Representative chemical structures include:

### PF-06928215 ((1R,2S)-2-[(7-oxo-5-phenyl-1H-pyrazolo[1,5-a]pyrimidine-3-carbonyl)amino]cyclohexane-1-carboxylic Acid)

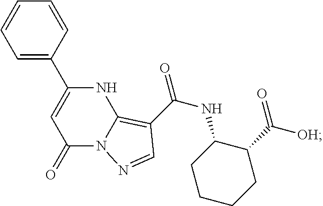 [SMILES: O=C(N[C@H]1CCCC[C@H]1C(=O)O)c1cnn2c(=O)cc(-c3ccccc3)[nH]c12]

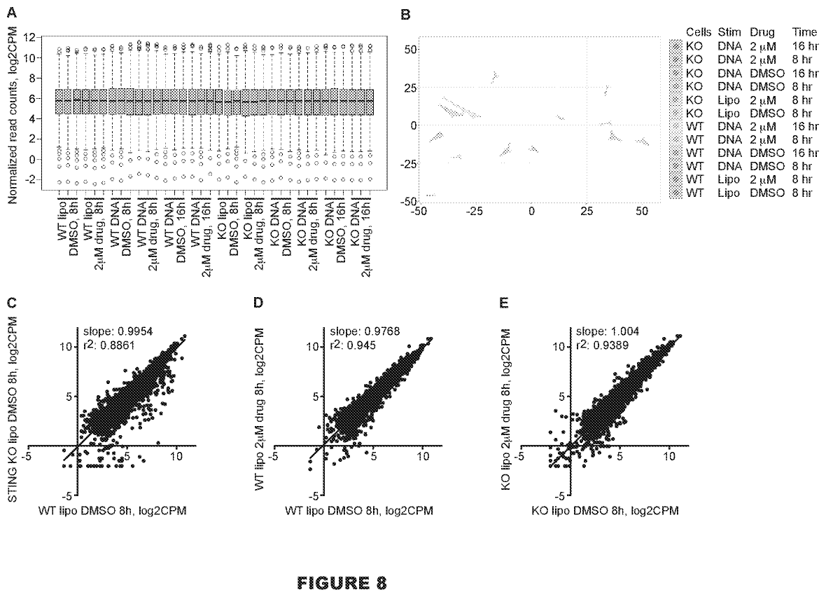

RU.521 (3-[1-(6,7-dichloro-1H-benzimidazol-2-yl)-5-hydroxy-3-methyl-pyrazol-4-yl]-3H-isobenzofuran-1-one; Supplemental FIG. 8, Nat Commun. 2017 Sep. 29; 8(1):750. doi: 10.1038/s41467-017-00833-9):

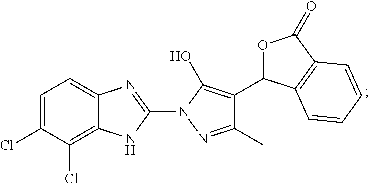 [SMILES: Cc1nn(-c2nc3ccc(Cl)c(Cl)c3[nH]2)c(O)c1C1OC(=O)c2ccccc21]

STING inhibitors disclosed in Nature. 2018 July; 559(7713):269-273. doi: 10.1038/s41586-018-0287-8. Epub 2018 Jul. 4, such as:

### C-170 (N-(4-butylphenyl)-5-nitrofuran-2-carboxamide)

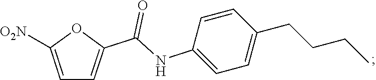 [SMILES: CCCCc1ccc(NC(=O)c2ccc([N+](=O)[O-])o2)cc1]

### C-171 (N-(4-hexylphenyl)-5-nitrofuran-2-carboxamide)

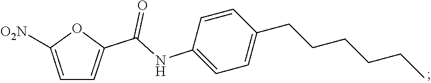 [SMILES: CCCCCCc1ccc(NC(=O)c2ccc([N+](=O)[O-])o2)cc1]

### H-151 (1-(4-ethylphenyl)-3-(1H-indol-3-yl)urea)

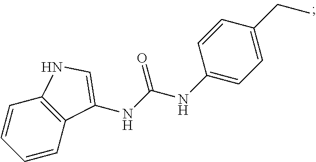 [SMILES: CCc1ccc(NC(=O)Nc2c[nH]c3ccccc23)cc1]

### H-151-AL (1-(4-ethynylphenyl)-3-(1H-indol-3-yl)urea)

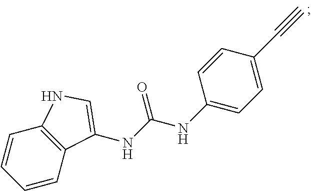 [SMILES: C#Cc1ccc(NC(=O)Nc2c[nH]c3ccccc23)cc1]

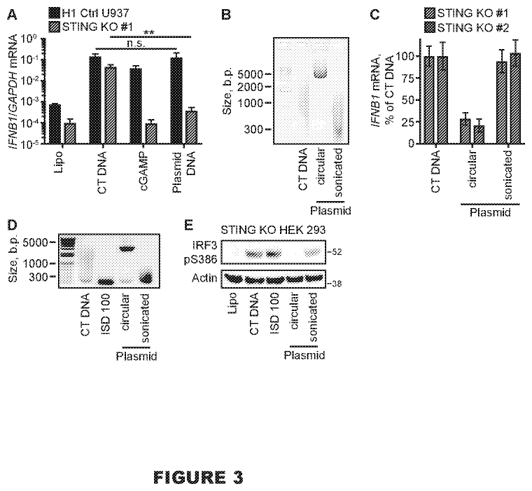

and
cGAS inhibitors described in PLos One 2017 Sep. 21; 12(9):e0184843. doi: 10.1371/journal. pone.0184843. eCollection 2017, FIG. 3, such as:

### 5-phenyltetrazolo[1,5-a]pyrimidin-7-ol (Compound 15)

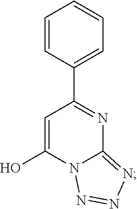 [SMILES: Oc1cc(-c2ccccc2)nc2nnnn12]

### 7-hydroxy-N-methyl-5-phenylpyrazolo[1,5-a]pyrimidine-3-carboxamide (Compound 16)

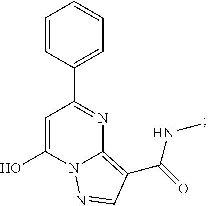 [SMILES: CNC(=O)c1cnn2c(O)cc(-c3ccccc3)nc12]

### 7-hydroxy-N-(2-hydroxyethyl)-5-phenylpyrazolo[1,5-a]pyrimidine-3-carboxamide (Compound 17)

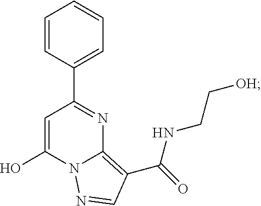 [SMILES: O=C(NCCO)c1cnn2c(O)cc(-c3ccccc3)nc12]

### (7-hydroxy-5-phenylpyrazolo[1,5-a]pyrimidine-3-carbonyl)glycine (Compound 18)

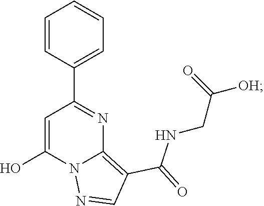 [SMILES: O=C(O)CNC(=O)c1cnn2c(O)cc(-c3ccccc3)nc12]

and

### (S)-7-hydroxy-N-(1-hydroxypropan-2-yl)-5-phenylpyrazolo[1,5-a]pyrimidine-3-carboxamide (Compound 19)

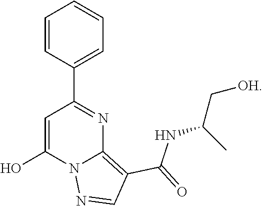 [SMILES: C[C@@H](CO)NC(=O)c1cnn2c(O)cc(-c3ccccc3)nc12]

Autoinflammatory diseases are caused by genetic mutations in molecules that are involved in regulating the innate immune response-a “hard wired” defense system that evolved to quickly recognize and act against infectious agents and other danger signals produced by our bodies. Autoimmune diseases are caused by the body's adaptive immune system developing antibodies to antigens that then attack healthy body tissues.

The methods disclosed herein can be used to treat any autoimmune or auto-inflammatory disease. Exemplary autoimmune diseases that can be treated, or development limited, using the methods of the invention include, but are not limited to Systemic lupus erythematosus (SLE), Discoid lupus, Cutaneous lupus, Sjogrens syndrome, Aicardi-Goutieres syndrome (AGS), pemphigoid (any type), Crohn's disease, endometriosis, fibromyalgia, glomerulonephritis, juvenile arthritis, type 1 diabetes, multiple sclerosis, psoriasis, rheumatoid arthritis, sarcoidosis, scleroderma, and ulcerative colitis. In specific embodiments, the autoimmune disease comprises Cutaneous lupus or scleroderma.

As used herein, “treat” or “treating” means accomplishing one or more of the following: (a) reducing the severity of the disease; (b) limiting or preventing development of symptoms, including flares, characteristic of the disease; (c) inhibiting worsening of symptoms characteristic of the disease; (d) limiting or preventing recurrence of the disease or symptoms in subjects that were previously symptomatic for.

In all embodiments disclosed herein, any level of inhibition of activity (such as expression, activity (such as kinase activity), and/or stability) is beneficial to treat the autoimmune disorder or the auto-inflammatory disorder. In various non-limiting embodiments, the inhibitors administered inhibit activity of the relevant target by at least 5%, 10%, 20%, 25%, 30%, 40%, 50%, 60%, 75%, 80%, 85%, 90%, or more compared to activity of the relevant target in a control (such as a base line level determined for the subject, a predetermined threshold level, etc.)

The “amount effective” of the administered therapeutic can be determined by an attending medical personnel based on all relevant factors. The therapeutic(s) may be the sole therapeutic(s) administered, or may be administered with other therapeutics as deemed appropriate by attending medical personnel in light of all circumstances.

The therapeutics may be administered singly, as mixtures of one or more compounds or in mixture or combination with other agents useful for treating such diseases and/or the symptoms associated with such diseases. The compounds may also be administered in mixture or in combination with agents useful to treat other disorders or maladies. The therapeutics may be administered in the form of compounds per se, or as pharmaceutical compositions comprising the therapeutic(s).

The amount of therapeutics(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular therapeutics(s), etc. Determination of an effective dosage of therapeutics(s) for a particular use and mode of administration is well within the capabilities of those skilled in the art. Effective dosages may be estimated initially from in vitro activity and metabolism assays. For example, an initial dosage of therapeutics for use in animals may be formulated to achieve a circulating blood or serum concentration of the therapeutics or metabolite active compound that is at or above an IC₅₀ of the particular therapeutics as measured in as in vitro assay. Calculating dosages to achieve such circulating blood or serum concentrations taking into account the bioavailability of the particular therapeutics via the desired route of administration is well within the capabilities of skilled artisans. Initial dosages of therapeutics can also be estimated from in vivo data, such as animal models.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active therapeutic, the bioavailability of the therapeutic, its metabolism kinetics and other pharmacokinetic properties, the mode of administration and various other factors, discussed above. Dosage amount and interval may be adjusted individually to provide plasma levels of the therapeutic(s) and/or active metabolite compound(s) which are sufficient to maintain therapeutic or prophylactic effect. For example, the therapeutics may be administered once per week, several times per week (e.g., every other day), once per day or multiple times per day, depending upon, among other things, the mode of administration, the specific indication being treated and the judgment of the prescribing medical personnel. In cases of local administration or selective uptake, such as local topical administration, the effective local concentration of therapeutic(s) and/or active metabolite therapeutic(s) may not be related to plasma concentration. Skilled artisans will be able to optimize effective dosages without undue experimentation.

In another aspect, the disclosure provides methods for monitoring therapy of a subject being treated for an autoimmune disease and/or an autoinflammatory disease, comprising

(a) determining a baseline level of HSPA8/HSC70 phosphorylation in a biological sample from the subject; and

(b) determining level of HSPA8/HSC70 phosphorylation in a biological sample from the subject 1 or more (2, 3, 4, 5, 6, or more times) after treatment for the autoimmune disease and/or an autoinflammatory disease,

wherein a decrease in HSPA8/HSC70 phosphorylation in the biological sample from the subject after treatment indicates efficacy of the therapy, and wherein an increase in HSPA8/HSC70 phosphorylation in the biological sample from the subject after treatment indicates that the therapy was ineffective.

The methods can be used to monitor therapy of a subject having any suitable autoimmune or auto-inflammatory disease, including but limited to those disclosed herein. In specific embodiments, the autoimmune disease comprises Cutaneous lupus or scleroderma.

Step (b) can be carried out any number of times over any suitable time frame as deemed appropriate by attending medical personnel. In another embodiment, when the method indicates that the therapy was ineffective, the method further comprises switching to a different therapy or increasing a dose of the therapeutic being administered. Any suitable methods for determining phosphorylation can be used, including but not limited to those disclosed herein. Any suitable biological sample from the subject may be used, including but not limited to blood samples, tissue or skin biopsies, etc. In one embodiment, determining the level of HSPA8/HSC70 phosphorylation comprises determining phosphorylation of serine 638 of human HSPA8/HSC70.

In another aspect, the disclosure provides methods for identifying compounds to treat autoimmune disease and/or autoinflammatory diseases, comprising identifying compounds that inhibit DNA-PK and/or HSPA8/HSC70 expression, activity, and/or stability. The methods can be used to identify compounds for treating any suitable autoimmune or auto-inflammatory disease, including but limited to those disclosed herein. In specific embodiments, the autoimmune disease comprises Cutaneous lupus or scleroderma. In one embodiment, the method comprises identifying compounds that inhibit DNA-PK phosphorylation of HSPA8/HSC70. In another embodiment, the method comprises identifying compounds that inhibit DNA-PK phosphorylation of serine 638 of HSPA8/HSC70.

In all of the methods disclosed herein, the subject may be any subject that has or is at risk of developing cancer. In one embodiment, the subject is a mammal, including but not limited to humans, dogs, cats, horses, cattle, etc.

In another aspect, the disclosure provides pharmaceutical compositions comprising:

(a) a DNA-PK inhibitor and/or an inhibitor of HSPA8/HSC70; and

(b) an inhibitor of cGAS expression, activity, and/or stability, and/or an inhibitor of STING expression, activity, and/or stability.

The pharmaceutical compositions can be used for any suitable purpose, including but not limited to treating autoimmune disorders and/or autoinflammatory diseases, such as by the methods of the disclosure. In one embodiment, the DNA-PK inhibitor and/or an inhibitor of HSPA8/HSC70 comprises a DNA-PK inhibitor. In another embodiment, the DNA-PK inhibitor comprises one or more of small molecule inhibitors of activity (such as kinase activity), antisense oligonucleotides directed against the DNA-PK DNA or mRNA; small interfering RNAs (siRNAs), short hairpin RNAs (shRNAs), microRNAs (miRNA) or small internally segmented interfering RNAs (sisiRNA) directed against the DNA-PK protein, DNA, or mRNA; DNA-PK antibodies, and aptamers that bind to DNA-PK. In a further embodiment, the DNA-PK inhibitor is a small molecule inhibitor. In one such embodiment, the DNA-PK small molecule inhibitor comprises one or more of NU-7441, M3814, Compound II, or Compound III (all shown below), or pharmaceutically acceptable salts, esters, or prodrugs thereof.

### NU-7441 (8-(dibenzo[b,d]thiophen-4-yl)-2-morpholino-4H-chromen-4-one)

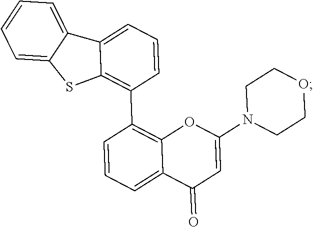 [SMILES: O=c1cc(N2CCOCC2)oc2c(-c3cccc4c3sc3ccccc34)cccc12]

### Nedisertib ((S)-[2-chloro-4-fluoro-5-(7-morpholin-4-ylquinazolin-4-yl)phenyl]-(6-methoxypyridazin-3-yl)methanol; M3814)

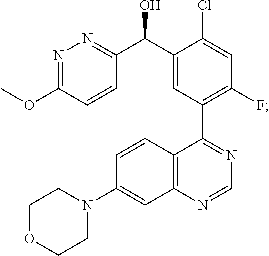 [SMILES: COc1ccc([C@@H](O)c2cc(-c3ncnc4cc(N5CCOCC5)ccc34)c(F)cc2Cl)nn1]

### Compound II (2-(Morpholin-4-yl)-benzo[h]chromen-4-one)

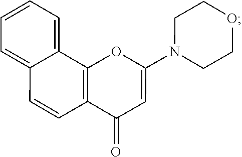 [SMILES: O=c1cc(N2CCOCC2)oc2c1ccc1ccccc12]

### Compound III (1-(2-hydroxy-4-morpholinophenyl)ethan-1-one)

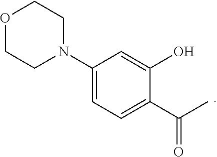 [SMILES: CC(=O)c1ccc(N2CCOCC2)cc1O]

In another embodiment, the pharmaceutical composition comprises an HSPA8/HSC70 inhibitor. In one such embodiment, the HSPA8/HSC70 inhibitor comprises a small molecule inhibitor of activity, antisense oligonucleotides directed against the HSPA8/HSC70 DNA or mRNA; small interfering RNAs (siRNAs), short hairpin RNAs (shRNAs), microRNAs (miRNA) or small internally segmented interfering RNAs (sisiRNA) directed against the HSPA8/HSC70 protein, DNA, or mRNA; HSPA8/HSC70 antibodies, aptamers that bind to HSPA8/HSC70, and any other chemical or biological compound that can interfere with HSPA8/HSC70 expression, activity, and/or stability.

In a further embodiment, the pharmaceutical composition comprises a cGAS inhibitor. In one such embodiment, the cGAS inhibitor comprises a small molecule cGAs inhibitor, antisense oligonucleotides directed against the cGAS DNA or mRNA; small interfering RNAs (siRNAs), short hairpin RNAs (shRNAs), microRNAs (miRNA) or small internally segmented interfering RNAs (sisiRNA) directed against the cGAS protein, DNA, or mRNA; cGAS antibodies, aptamers that bind to cGAS, and any other chemical or biological compound that can interfere with cGAS expression, activity, and/or stability. In another embodiment, the cGAS inhibitor comprises any cGAS inhibitors, such as PF-06928215, disclosed in PLos One 2017 Sep. 21; 12(9):e0184843. doi: 10.1371/journal. pone.0184843. eCollection 2017 and/or RU.521 (Nat Commun. 2017 Sep. 29; 8(1):750. doi: 10.1038/s41467-017-00833-9), or pharmaceutically acceptable salts, esters, or prodrugs thereof.

In another embodiment, the pharmaceutical composition comprises a STING inhibitor. In one such embodiment, the STING inhibitor comprises a small molecule STING inhibitor, antisense oligonucleotides directed against the STING DNA or mRNA; small interfering RNAs (siRNAs), short hairpin RNAs (shRNAs), microRNAs (miRNA) or small internally segmented interfering RNAs (sisiRNA) directed against the STING protein, DNA, or mRNA; STING antibodies, aptamers that bind to STING, and any other chemical or biological compound that can interfere with STING expression, activity, and/or stability. In another embodiment, the STING inhibitor comprises one or more STING inhibitors disclosed in Nature. 2018 July; 559(7713):269-273. doi: 10.1038/s41586-018-0287-8. Epub 2018 Jul. 4.

Representative chemical structures include:

### PF-06928215 (1R,2S)-2-[(7-oxo-5-phenyl-1H-pyrazolo[1,5-a]pyrimidine-3-carbonyl)amino]cyclohexane-1-carboxylic Acid)

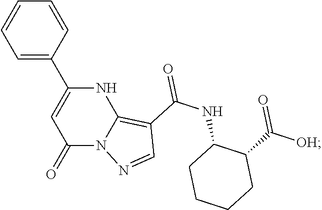 [SMILES: O=C(N[C@H]1CCCC[C@H]1C(=O)O)c1cnn2c(=O)cc(-c3ccccc3)[nH]c12]

RU.521 (3-[1-(6,7-dichloro-1H-benzimidazol-2-yl)-5-hydroxy-3-methyl-pyrazol-4-yl]-3H-isobenzofuran-1-one; Supplemental FIG. 8, Nat Commun. 2017 Sep. 29; 8(1):750. doi: 10.1038/s41467-017-00833-9):

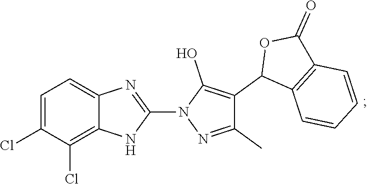 [SMILES: Cc1nn(-c2nc3ccc(Cl)c(Cl)c3[nH]2)c(O)c1C1OC(=O)c2ccccc21]

STING inhibitors disclosed in Nature. 2018 July; 559(7713):269-273. doi: 10.1038/s41586-018-0287-8. Epub 2018 Jul. 4, such as:

### C-170 (N-(4-butylphenyl)-5-nitrofuran-2-carboxamide)

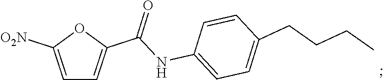 [SMILES: CCCCc1ccc(NC(=O)c2ccc([N+](=O)[O-])o2)cc1]

### C-171 (N-(4-hexylphenyl)-5-nitrofuran-2-carboxamide)

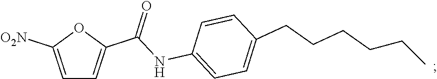 [SMILES: CCCCCCc1ccc(NC(=O)c2ccc([N+](=O)[O-])o2)cc1]

### H-151 (1-(4-ethylphenyl)-3-(1H-indol-3-yl)urea)

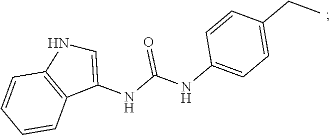 [SMILES: CCc1ccc(NC(=O)Nc2c[nH]c3ccccc23)cc1]

### H-151-AL (1-(4-ethynylphenyl)-3-(1H-indol-3-yl)urea)

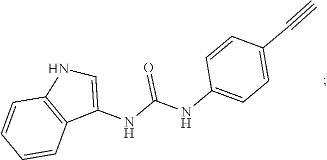 [SMILES: C#Cc1ccc(NC(=O)Nc2c[nH]c3ccccc23)cc1]

and
cGAS inhibitors described in PLos One 2017 Sep. 21; 12(9):e0184843. doi: 10.1371/journal. pone.0184843. eCollection 2017, FIG. 3, such as:

### 5-phenyltetrazolo[1,5-a]pyrimidin-7-ol (Compound 15)

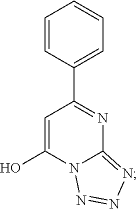 [SMILES: Oc1cc(-c2ccccc2)nc2nnnn12]

### 7-hydroxy-N-methyl-5-phenylpyrazolo[1,5-a]pyrimidine-3-carboxamide (Compound 16)

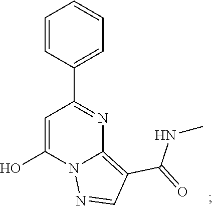 [SMILES: CNC(=O)c1cnn2c(O)cc(-c3ccccc3)nc12]

### 7-hydroxy-N-(2-hydroxyethyl)-5-phenylpyrazolo[1,5-a]pyrimidine-3-carboxamide (Compound 17)

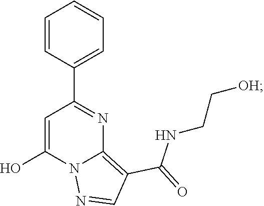 [SMILES: O=C(NCCO)c1cnn2c(O)cc(-c3ccccc3)nc12]

### (7-hydroxy-5-phenylpyrazolo[1,5-a]pyrimidine-3-carbonyl)glycine (Compound 18)

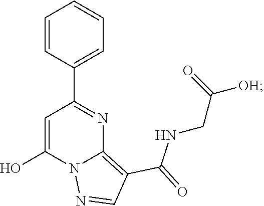 [SMILES: O=C(O)CNC(=O)c1cnn2c(O)cc(-c3ccccc3)nc12]

and

### (S)-7-hydroxy-N-(1-hydroxypropan-2-yl)-5-phenylpyrazolo[1,5-a]pyrimidine-3-carboxamide (Compound 19)

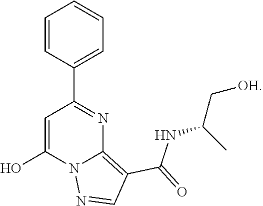 [SMILES: C[C@@H](CO)NC(=O)c1cnn2c(O)cc(-c3ccccc3)nc12]

In a further embodiment, the DNA-PK inhibitor, the HSPA8/HSC70 inhibitor, the cGAS inhibitor, and the STING inhibitor are not inhibitors expressed by non-recombinant viruses. In another embodiment, the DNA-PK inhibitor, the HSPA8/HSC70 inhibitor, the cGAS inhibitor, and the STING inhibitor are not naturally occurring inhibitors.

The pharmaceutical compositions may further comprise a pharmaceutically acceptable carrier, excipient or diluent. The exact nature of the carrier, excipient or diluent will depend upon the desired use for the composition, and may range from being suitable or acceptable for veterinary uses to being suitable or acceptable for human use.

Pharmaceutical compositions comprising the therapeutic(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically. The therapeutics may be formulated in the pharmaceutical composition per se, or in the form of a hydrate, solvate, N-oxide or pharmaceutically acceptable salt. Typically, such salts are more soluble in aqueous solutions than the corresponding free acids and bases, but salts having lower solubility than the corresponding free acids and bases may also be formed. Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the therapeutic(s) may be formulated as solutions, gels, ointments, creams, suspensions, etc. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active therapeutic(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active therapeutic(s) may be dried by any technique, such as lyophilization, and reconstituted prior to use.

For transmucosal administration, penetrants appropriate to the barrier to be permeated are used in the formulation.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by several methods, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the therapeutics. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the therapeutics can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the therapeutics may be formulated as a solution, emulsion, suspension, etc. suitable for administration to the eye. A variety of vehicles are suitable for administering compounds to the eye.

For prolonged delivery, the therapeutics can be formulated as a depot preparation for administration by implantation or intramuscular injection. The therapeutics may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the therapeutics for percutaneous absorption may be used. To this end, permeation enhancers may be used to facilitate transdermal penetration of the therapeutics.

Alternatively, other pharmaceutical delivery systems may be employed. Liposomes and emulsions are examples of delivery vehicles that may be used to deliver therapeutics. Certain organic solvents such as dimethyl sulfoxide (DMSO) may also be employed.

The therapeutics described herein, or compositions thereof, will generally be used in an amount effective to achieve the intended result, for example in an amount effective to treat or prevent the particular disease being treated, and dosage forms of the compositions generated accordingly.

### Examples

### Summary

Detection of intracellular DNA by the cGAS-STING pathway activates a type I interferon-mediated innate immune response that protects from virus infection and can be harnessed to promote anti-tumor immunity. Whether there are additional DNA sensing pathways, and how such pathways might function, remains controversial. We show here that humans—but not mice—have a second, potent, STING-independent DNA sensing pathway that is blocked by the E1A viral oncogene of human adenovirus 5. We identify human DNA-PK as the sensor of this pathway and demonstrate that DNA-PK kinase activity drives a robust and broad antiviral response. We discover that the heat shock protein HSPA8/HSC70 is a unique target of the DNA-PK SIDSP. Finally, we demonstrate that detection of foreign DNA and DNA damage trigger distinct modalities of DNA-PK activity. These findings reveal the existence, sensor, unique target, and viral antagonists of a STING-independent DNA sensing pathway (SIDSP) in human cells.

### Introduction

The cGAS-STING DNA sensing pathway has emerged as a key component of the innate immune response that is important for antiviral immunity (1), contributes to specific autoimmune diseases (2), and mediates important aspects of antitumor immunity (3). cGAS binds to double-stranded DNA and catalyzes the formation of cyclic GMP-AMP (cGAMP; 4, 5), a diffusible cyclic dinucleotide that activates the endoplasmic adaptor protein STING (6). Activated STING then serves as a platform for the inducible recruitment of the TBK1 kinase, which phosphorylates and activates the transcription factor IRF3, leading to the induction of the type I interferon mediated antiviral response (7).

Here, we report the unexpected finding that the E1A oncogene of human adenovirus 5 blocks two distinct DNA sensing pathways in human cells: the well-known cGAS-STING pathway (11), and a second, STING-independent DNA sensing pathway (SIDSP). We identify the DNA damage response protein DNA-PK as the sensor of the SIDSP, along with the heat shock protein HSPA8 as a unique SIDSP target. We show that the SIDSP is potently activated in human and primate cells, but it is weak or absent from mouse cells. Our findings demonstrate that human cells have a second DNA sensing pathway, with implications for host defense, autoimmunity, and anti-tumor immunity.

### Results

### Human Adenovirus 5 E1A Blocks Two DNA Sensing Pathways in Human Cells

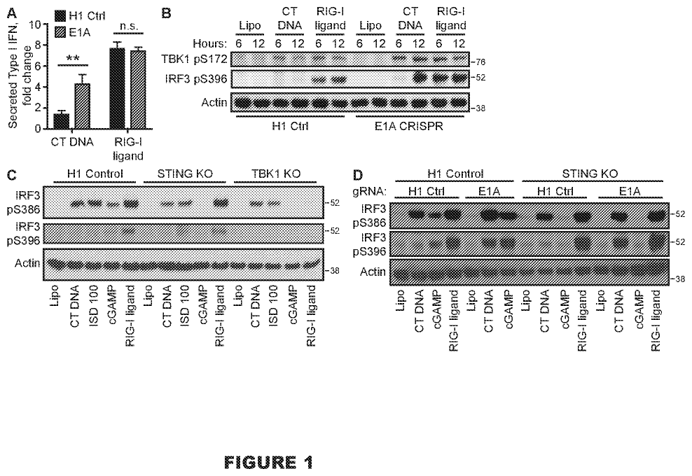

We previously demonstrated that the viral oncogenes of the DNA tumor viruses are potent antagonists of the cGAS-STING DNA sensing pathway (11). We sought to define the mechanism of this antagonism, focusing on the E1A oncogene of human adenovirus 5, which is constitutively expressed in human HEK 293 cells and is responsible for their transformation. As shown previously (11), we found that HEK 293 cells mounted a robust type I IFN response to RIG-I ligand, but not to transfected calf thymus (CT) DNA, and that CRISPR-mediated disruption of E1A restored the DNA-activated IFN response (FIG. 1A). We monitored key early events in the antiviral signaling pathways that are activated by cGAS-STING and the RNA-activated RIG-I-MAVS pathways: the activation and autophosphorylation of the kinase TBK1, and the subsequent TBK1-dependent phosphorylation of the IRF3 transcription factor on several residues that leads to IRF3 dimerization, nuclear translocation, and transcription of the type I IFN genes (7). In E1A-expressing control HEK 293 cells, we found that CT DNA transfection resulted in detectable phosphorylation of TBK1 on serine 172, but phosphorylation of IRF3 on serine 396 was impaired compared to RIG-I ligand-activated IRF3 phosphorylation (FIG. 1B). Disruption of E1A restored DNA-activated IRF3 S396 phosphorylation (FIG. 1B). These data suggested that E1A blocks IRF3 activation at a step between TBK1 activation and IRF3 phosphorylation, leading us to evaluate IRF3 activation more thoroughly. To do this, we used an antibody to detect phosphorylation of IRF3 on serine 386, which is essential for IRF3 dimerization (7). To test for the specificity of the response, we used lentiCRISPR to generate clonal lines of STING- and TBK1-deficient HEK 293 cells, validating disruption of the genes by DNA sequencing and western blot (FIG. 8). We found that transfection of either DNA or the direct STING agonist cGAMP resulted in robust IRF3 S386 phosphorylation in E1A-expressing control HEK 293 cells, but IRF3 S396 phosphorylation was impaired (FIG. 1C). This IRF3 S386 phosphorylation after cGAMP treatment was absent from STING- and TBK1-deficient cells, confirming that cGAMP-induced IRF3 activation was both STING- and TBK1-dependent. Surprisingly, however, DNA-activated IRF3 S386 phosphorylation was intact in both STING-deficient and TBK1-deficient HEK 293 cells (FIG. 1C). Disruption of E1A restored both STING-dependent IRF3 S396 phosphorylation in response to cGAMP and STING-independent IRF3 S396 phosphorylation in response to DNA. These data demonstrate that E1A blocks the DNA-activated antiviral response at a late step, after initiation of IRF3 phosphorylation but before its completion. Moreover, E1A unexpectedly blocks two distinct antiviral responses in HEK 293 cells, one STING-dependent and one STING-independent.

### A STING-Independent DNA Sensing Pathway (SIDSP) in Human Cells

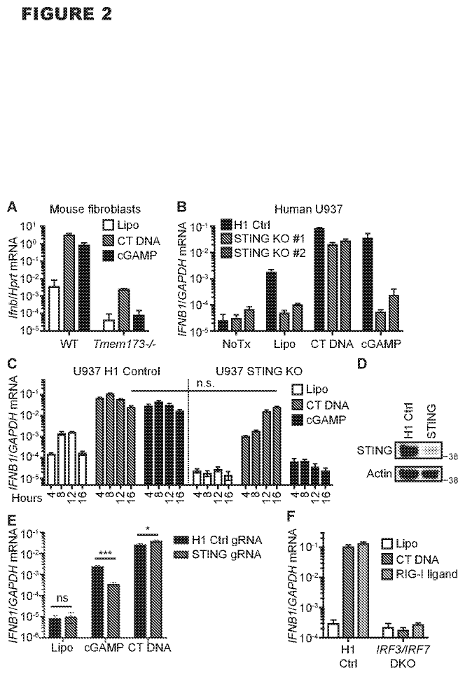

To explore these two DNA sensing pathways in more detail, we turned to additional mouse and human cell lines. We first confirmed that the type I IFN response to transfected CT DNA was STING-dependent in primary mouse fibroblasts at the peak of the response four hours post transfection: both DNA- and cGAMP-activated IFN responses were reduced by 99.9% at this time point in STING-deficient fibroblasts (FIG. 2A). We generated two independent clonal lines of STING-deficient human U937 monocytes, a well-characterized human lymphoma cell line. We transfected these cells with DNA or cGAMP and measured IFNB1 transcription by quantitative RT-PCR 16 hours after transfection. As expected, both STING-deficient U937 clones failed to respond to cGAMP (FIG. 2B). However, DNA transfection of STING-deficient U937 cells activated a potent type I IFN response (FIG. 2B). We then performed a time course analysis of cGAMP- and DNA-activated IFN responses, comparing control U937 cells to STING-deficient cells. Control U937 cells responded to both DNA and cGAMP transfection with robust IFNB1 transcription that peaked at 8 hours. STING-deficient U937 cells failed to respond to cGAMP, but they activated a potent antiviral response to DNA that was delayed by several hours, peaking at 16 hours with IFNB1 mRNA levels that were indistinguishable from those of control cells at this same time point (FIG. 2C). We next tested whether the antiviral response to DNA was dependent on the transcription factors IRF3 and IRF7, which together are essential for the IFN response to all other known nucleic acid detection pathways (12). To do this, we used a previously described clonal line of IRF3/IRF7 double knockout human THP1 monocytes (13). We found that the potent IFNB1 transcription in response to both DNA and RNA was completely IRF3/7-dependent at 16 hours post transfection (FIG. 2D). These findings reveal three important points about the DNA-activated antiviral response. First, and consistent with dozens of prior studies, the antiviral response to DNA in mouse cells is nearly entirely STING-dependent (9). Second, unlike mouse cells, human cells possess a robust STING-independent DNA sensing pathway that is delayed relative to the cGAS-STING pathway. Third, IRF3 and IRF7 are essential for both DNA-activated antiviral responses in human cells. Thus, human cells—but not mouse cells—have a robust STING-independent DNA sensing pathway (SIDSP).

Using our control and STING-deficient U937 cells to genetically separate the cGAS-STING pathway from the SIDSP, we evaluated the structural features of the DNA ligands that triggered these pathways. cGAS activation is mediated by its binding to the sugar phosphate backbone of double-stranded DNA in a sequence-independent manner (14, 15). Accordingly, control U937 cells mounted an equally robust antiviral response to both sheared CT DNA and circular plasmid DNA (FIG. 3A). However, STING KO U937 cells responded potently to CT DNA but not to plasmid DNA (FIG. 3A). We found that sonication of the plasmid DNA prior to transfection restored the IFN response in STING KO U937 cells to levels that were identical to those activated by CT DNA (FIGS. 3, B and C). Similarly, phosphorylation of IRF3 serine 386 in STING KO HEK 293 cells was potently activated by CT DNA, annealed 100 base-pair DNA oligos (ISD100), and sonicated plasmid DNA, but not by circular plasmid DNA (FIGS. 3, D and E). Thus, the cGAS-STING pathway and the SIDSP are activated by different features of DNA: cGAS detects the backbone of dsDNA, whereas the SIDSP detects DNA ends.

### Human DNA-PK is Essential for the SIDSP

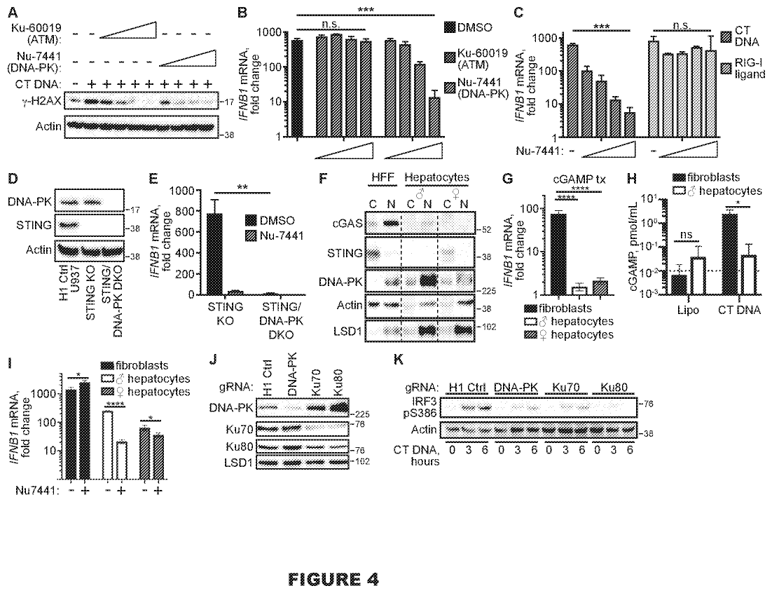

Our finding that the activation of the SIDSP requires exposed DNA ends led us to consider two key DNA damage response pathways that are activated by DNA ends: the Ataxia-Telangiectasia Mutated kinase (ATM) pathway that is important for homology-dependent DNA repair, and the DNA-dependent Protein Kinase (DNA-PK) pathway that mediates non-homologous DNA end joining (NHEJ; 16). We transfected STING KO U937 cells with CT DNA in the presence of well-characterized chemical inhibitors of the kinase activities of ATM (Ku-60019; 17) or DNA-PK (Nu-7441; 18). Both of these inhibitors reduced the DNA-activated phosphorylation of the histone H2AX on serine 139 (γ-H2AX) in a concentration-dependent manner, confirming their activity in these cells (FIG. 4A). However, we found that the SIDSP was potently blocked by the DNA-PK inhibitor but unaffected by the ATM inhibitor (FIG. 4B). Moreover, the DNA-PK inhibitor Nu-7441 blocked the DNA-activated antiviral response but had no effect on the RNA-activated RIG-I-MAVS pathway (FIG. 4C).

We next used lentiCRISPR to simultaneously target U937 cells with guide RNAs targeting STING and the catalytic subunit of DNA-PK (DNA-PKcs), which is encoded by the PRKDC gene. DNA-PK-targeted cells were severely compromised for growth relative to control cells, as has been previously reported (19), but we managed to generate a clonal line of U937 cells doubly deficient for STING and DNA-PK, verified by western blot and DNA sequencing, together with a third clonal line of STING KO U937 cells (FIG. 4D). Consistent with the chemical inhibitor data, we found that STING/DNA-PK DKO U937 cells were profoundly impaired in their IFN response to DNA (FIG. 4E).

The activation of DNA-PK requires the Ku70 and Ku80 cofactors that are responsible for DNA end binding and recruitment of DNA-PKcs to damaged DNA (16). We attempted to generate clonal lines of U937 cells deficient for Ku70 and Ku80 but we were not able to recover live knockout cells, likely because they are essential genes in human somatic cells (20, 21). We therefore employed a transient lentiCRISPR approach in STING KO HEK 293 cells to target the XRCC6 (Ku70) and XRCC5 (Ku80) genes at the population level. Three days after selection of transduced cells in puromycin, we observed reduced levels of DNA-PKcs, Ku70, and Ku80 proteins in HEK 293 cells targeted with the respective guide RNAs (FIG. 4F). Moreover, targeting Ku70 resulted in reduced expression of Ku80 protein and vice versa, consistent with prior data demonstrating that Ku70 is required for Ku80 protein stability (22). We found that IRF3 S386 phosphorylation in response to DNA transfection was reduced in cells targeted for DNA-PKcs, Ku70, and Ku80, suggesting that all three of these components of the DNA-PK complex are essential for SIDSP activation (FIG. 4G). Together, our data provide pharmacological and genetic evidence that DNA-PK is essential for the SIDSP in human cells.

### The DNA-PK SIDSP Activates a Broad, Potent Antiviral Response

To define the nature of the transcriptional changes in the DNA-PK SIDSP beyond the canonical antiviral cytokine IFNβ, we performed a global mRNA-Seq analysis in WT and STING KO cells, evaluating the changes following DNA transfection and the effect of the DNA-PK inhibitor Nu-7441 on this response. After mapping to the human transcriptome, normalizing read counts across all samples, and removing features with fewer than 10 mean counts per million (CPM), our dataset revealed tight concordance among the three biological replicates within each condition and differential clustering of each condition relative to all others (FIGS. 8A and B). A direct comparison of WT and STING KO U937 cells treated with transfection reagent alone revealed largely similar basal gene expression levels, demonstrating that the clonal line of STING KO U937 cells was comparable to control cells (FIG. 8C). Moreover, we found that treatment with Nu-7441 in the absence of DNA stimulation had no significant effect on gene expression in either WT or STING KO cells (FIGS. 8, D and E), establishing the baseline conditions used for comparison to the DNA-treated samples within each genotype.

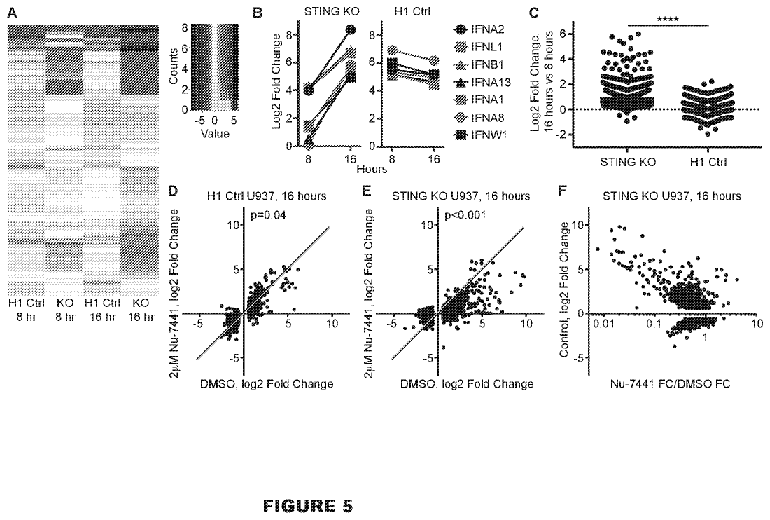

We compared DNA-activated WT and STING KO samples at 8 and 16 hours post-transfection to their respective transfection reagent alone controls, in the presence of DMSO or 204 Nu-7441. We focused first on the interferon-mediated antiviral response, objectively defined here by compiling genes in this category delineated by Gene Ontology Consortium terms. We compiled a list of antiviral response genes with a fold change of greater than 1.5 and a false discovery rate (FDR) of <0.05 in any one of the comparisons. A heat map of these 124 differentially expressed genes revealed a broad, potent, and overlapping antiviral program triggered by DNA in both WT and STING KO cells (FIG. 5A). Focusing on the seven IFN genes that were significantly induced in either WT or KO cells, we found that DNA transfection activated broad IFN expression in both WT and STING KO cells (FIG. 5B). Moreover, and consistent with the delayed IFNB1 response in STING KO cells (FIG. 2C), we found that expression of all IFNs increased between 8 and 16 hours in the STING KO cells, whereas these same genes plateaued or decreased in expression between 8 and 16 hours in WT cells (FIG. 5B). This delay in SIDSP-mediated gene expression held true when objectively comparing all upregulated genes in STING KO and WT cells. Specifically, 912/926 (98.5%) of upregulated genes in STING KO cells continued to increase in gene expression between 8 and 16 hours, with a larger relative increase compared to WT cells (FIG. 5C). Thus, the SIDSP activates a potent, broad gene expression program that is delayed relative to the DNA-activated antiviral response in WT human cells.

We next quantitated the effect of the Nu-7441 DNA-PK inhibitor on global gene expression in WT and STING KO cells. We plotted the fold change values of all differentially expressed genes at 16 hours post DNA transfection, comparing vehicle-treated cells to those treated with 2 μM Nu-7441. In WT cells, we found that Nu-7441 had a mild inhibitory effect on the expression of 718/1024 (70.1%) of differentially expressed genes (FIG. 5D), suggesting that at least some of the DNA-activated gene expression in WT human cells reflects the combined contributions of cGAS-STING and the SIDSP. Strikingly, we found that Nu-7441 had an inhibitory effect on 1254/1327 (94.5%) of differentially expressed genes in STING KO cells, including both upregulated and downregulated transcripts (FIG. 5E). Moreover, a plot of the fold change in vehicle-treated STING KO cells versus the inhibitory effect of Nu-7441 revealed that the most differentially expressed genes tended to be those most affected by the drug (FIG. 5F).

These mRNA-Seq data reveal a number of important features of the DNA-PK SIDSP. First, the SIDSP is a broad and potent antiviral response that results in significant changes in expression of over a thousand human genes. Second, global gene expression in the SIDSP is delayed relative to the DNA-activated antiviral response in WT human cells, highlighting kinetic differences of antiviral signaling that will be interesting to explore in the future. Third, the Nu-7441 inhibitor of DNA-PK kinase activity influences the vast majority of differential gene expression in the SIDSP, as well as a fraction of gene expression in WT cells. Thus, DNA-PK kinase activity is at the apex of the SIDSP, strongly suggesting that it is the primary sensor of this pathway rather than an incidentally activated peripheral component of a distinct pathway. Importantly, these data provide a clear rationale and framework for exploring the utility of DNA-PK inhibitors in IFN-mediated human autoimmune and autoinflammatory disorders.

### Human HSPA8/HSC70 is a Target of the DNA-PK SIDSP

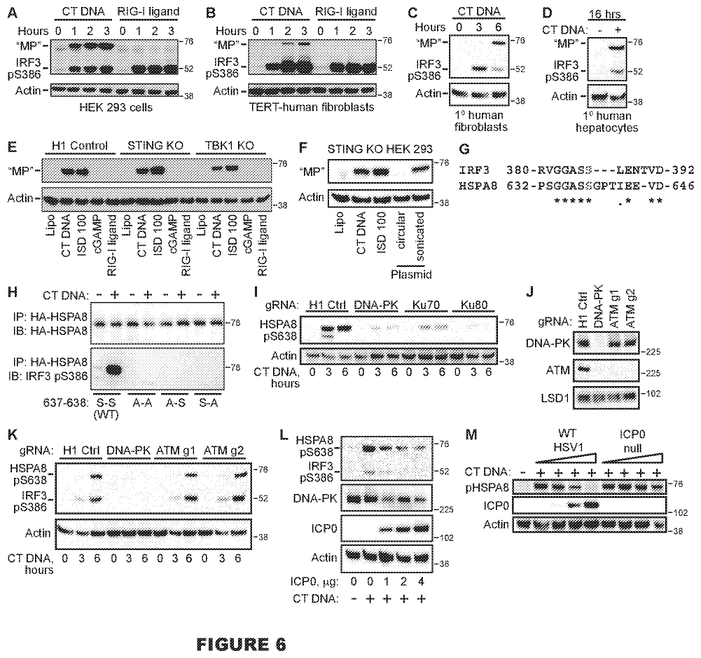

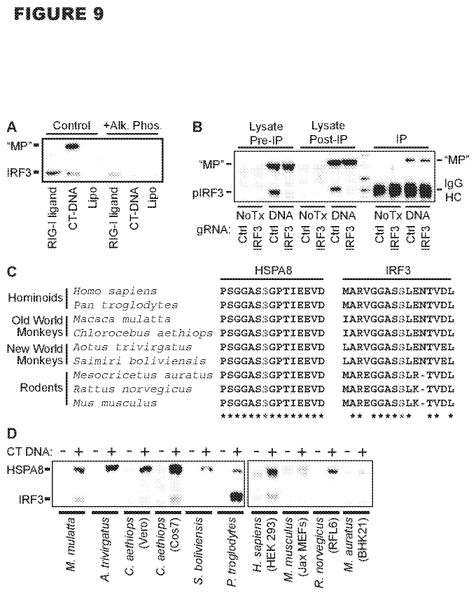

In our studies of E1A antagonism of IRF3 phosphorylation, we found that the antibody raised against IRF3 pS386 detected a second protein that was approximately 20 kilodaltons larger than IRF3 in DNA-activated HEK 293 cells (FIG. 6A). We found that this signal was sensitive to phosphatase treatment (FIG. 9A), thus identifying a novel, cross-reactive phosphoprotein that we named “Mystery Protein” (MP). We detected the DNA-activated phosphorylation of MP in all human cell lines examined, including HeLa cells (FIG. 6B), TERT-immortalized human fibroblasts (FIG. 6C), and primary human fibroblasts (FIG. 6D). Three key features of MP matched those that we defined for the SIDSP and led us to study it in more detail. First, MP appeared only in response to DNA, not RIG-I ligand or cGAMP (FIG. 6, A-E). Second, DNA-activated MP phosphorylation was independent of both STING and TBK1 (FIG. 6E). Finally, MP was phosphorylated in response to DNA ends but not circular plasmid DNA (FIG. 6F).

To identify MP, we used the IRF3 pS386 antibody for immunoprecipitation of HEK 293 cell extracts, followed by trypsin digest and mass spectrometry analysis of recovered peptides. To facilitate the identification of MP, we also generated IRF3-deficient HEK 293 cells using lentiCRISPR. Importantly, MP was still robustly phosphorylated after transfection of these IRF3-targeted cells with DNA, demonstrating that MP was not an unusual, slower migrating isoform of IRF3 itself, and that IRF3 was not required for MP phosphorylation (FIG. 9B). We found that the IRF3 pS386 antibody recovered detectable MP from lysates of DNA-transfected cells (FIG. 9B).

Among the peptides identified by mass spectrometry that were specifically enriched by IP with IRF3 pS386 antibody compared to control antibody, one protein in particular caught our attention. Heat shock protein A8 (HSPA8), also known as heat shock cognate 70 (HSC70), matched the predicted mass of MP at ˜73 kilodaltons. Most intriguingly, we noted a sequence at the extreme C terminus of HSPA8 that corresponds precisely to the sequence adjoining 5386 in IRF3, suggesting a probable explanation for cross-reactivity of the antibody (FIG. 6G). HSPA8 is an abundant, constitutively expressed member of the heat shock protein (HSP) family of chaperones that participate in the folding of cellular proteins into their native states, either after synthesis on the ribosome or after stress-induced unfolding (23).

To test whether MP was HSPA8, we generated expression vectors for hemagglutinin (HA) epitope-tagged human HSPA8 and three mutants in which one or both serines at positions 637 and 638 were mutated to alanines. We transfected each of these constructs into HEK 293 cells, waited 24 hours, and then transfected the cells with CT DNA for three hours before immunoprecipitation of the HA-tagged proteins and blotting for IRF3 pS386. We found that the IRF3 pS386 antibody robustly detected the WT HSPA8 protein after DNA transfection, but it failed to detect the single or double alanine-substituted mutant HSPA8 proteins (FIG. 6H). Thus, the IRF3 pS386 antibody detects phosphorylated human HSPA8, and both serines 637 and 638 of HSPA8 are essential for antibody binding. Because the IRF3 pS386 antibody was raised against a phosphopeptide in which only 5386 was phosphorylated, and because 5638 of HSPA8 aligns with S386 of IRF3, we suggest that HSPA8 is phosphorylated, at minimum, on S638 in response to DNA detection. Importantly, IRF3 is also known to be phosphorylated on S385 upon activation (7), so it is possible that HSPA8 is additionally phosphorylated on S637.

Similar to the data presented for IRF3 (FIG. 4F), we found that DNA-PK, Ku70, and Ku80 were all essential for the robust phosphorylation of HSPA8 on S638. Next, we compared the effect of DNA-PK and ATM disruption on phosphorylation of IRF3 and HSPA8. We confirmed previous findings that targeting DNA-PK resulted in a loss of ATM protein expression (24), and we found that disruption of ATM did not affect DNA-PK protein levels (FIG. 6J). Consistent with the pharmacological data in U937 monocytes (FIG. 4B), we observed that DNA-PK was essential for both IRF3 and HSPA8 phosphorylation after DNA transfection in HEK 293 cells, but loss of ATM had no effect on this response (FIG. 6K). Finally, we evaluated the effect of the ICP0 ubiquitin ligase of herpes simplex virus 1 (HSV-1), which targets DNA-PK for degradation (25), on activation of the SIDSP. We found that ICP0 expression in STING KO HEK 293 cells blocked DNA-activated phosphorylation of IRF3 and HSPA8 in a dose-dependent manner, revealing a second viral antagonist of the SIDSP (FIG. 6L). Thus, we have identified phosphorylation of human HSPA8 on serine 638 as a novel target of the DNA-PK SIDSP. Phosphorylation of the C-terminus of HSPA8 has been proposed to modulate its interactions with cochaperone proteins (26), but inducible, site-specific phosphorylation of HSPA8 has not been reported previously.

### HSPA8 Delineates the Antiviral Modality of Human DNA-PK

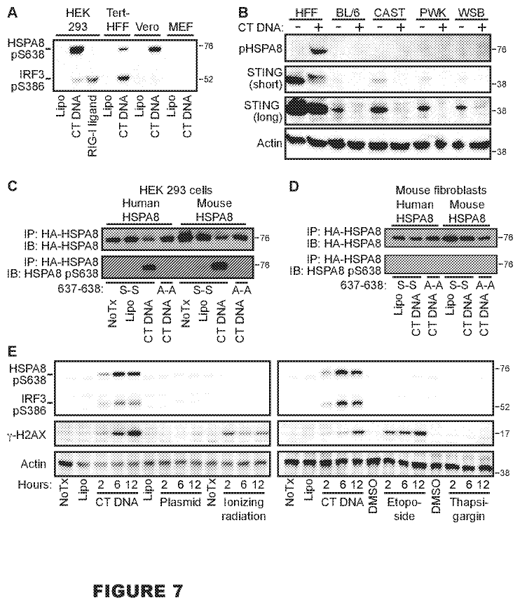

We noted that the amino acids surrounding serine 638 of HSPA8 are completely conserved across mammalian evolution, unlike those surrounding IRF3 (FIG. 9C). We took advantage of this conservation to test whether we could detect activation of the DNA-PK SIDSP in cell lines from primates and rodents, using HSPA8 phosphorylation as a convenient marker. We found that HSPA8 phosphorylation was robustly activated after DNA transfection in cells from all primate species tested and in rat cells, but that mouse fibroblasts failed to activate HSPA8 phosphorylation (FIGS. 7A and 12D). We then introduced epitope-tagged mouse HSPA8 into human HEK 293 cells, and human HSPA8 into mouse fibroblasts. Mouse HSPA8 was robustly phosphorylated in human cells, but neither human nor mouse HSPA8 became phosphorylated in mouse fibroblasts after DNA transfection (FIGS. 7, B and C). Together with the well documented observation that nearly all of the IFN response to DNA in mouse cells is STING-dependent (FIG. 2A), our data suggest that the DNA-PK SIDSP is present in humans, primates, and rats, but absent or severely impaired in mouse cells.

We next tested for activation of the SIDSP in response to DNA damage, which potently triggers activation of DNA-PK (16). We treated STING KO HEK 293 cells with CT DNA, plasmid DNA, ionizing radiation, or the topoisomerase-II inhibitor etoposide, monitoring activation of both IRF3 and HSPA8 phosphorylation up to 12 hours after treatment. As shown in FIGS. 3E, 4A, and 6F, we found that CT DNA, but not circular plasmid DNA, induced robust phosphorylation of both IRF3 and HSPA8, together with potent activation of H2AX S139 phosphorylation (FIG. 7D). However, and intriguingly, neither ionizing radiation nor etoposide activated IRF3 or HSPA8 phosphorylation, despite robust H2AX S139 phosphorylation and the well characterized activation of DNA-PK by each of these DNA damaging agents (FIG. 7D; 16). As a control, we used thapsigargin, which induced a potent endoplasmic reticulum (ER) stress response, but not IRF3, HSPA8, or H2AX phosphorylation (FIG. 7D). Together, our data suggest two distinct modalities of DNA-PK activation. First, the well-characterized role of DNA-PK in response to DNA damage, which involves coordination of the NHEJ repair machinery, does not activate DNA-PK-dependent phosphorylation of IRF3 or HSPA8. Second, the DNA-PK-dependent response to foreign DNA triggers both IRF3 and HSPA8 phosphorylation. Thus, the SIDSP activates unique targets of DNA-PK in a manner that is distinct from that triggered by DNA damage.

### DISCUSSION

We have identified DNA-PK as the sensor of a potent, STING-independent DNA sensing pathway (SIDSP) that is present in human cells but weak or absent from mouse cells. We identify two DNA virus-encoded antagonists of the DNA-PK SIDSP, and we show that a small molecule inhibitor of DNA-PK kinase activity potently reduces the robust and broad transcriptional response triggered by foreign DNA in human cells. Finally, we present evidence that the DNA-PK SIDSP includes unique targets that are triggered only by foreign DNA and not by DNA damage. The existence of a second DNA sensing pathway that is present in human cells but not mouse cells has important implications for our understanding of antiviral immunity, for treating autoimmune diseases, and for the possibility of harnessing this pathway to enhance immune responses to tumors.

We found that the Nu-7441 DNA-PK inhibitor potently reduced nearly all gene expression triggered by the SIDSP, demonstrating that DNA-PK kinase activity drives the SIDSP transcriptional response.

We identified serine 638 of HSPA8 as a unique and specific target of the DNA-PK SIDSP in human cells. We used the conservation of HSPA8 among mammals as a means to explore the activation of the DNA-PK SIDSP in primates and rodents. Consistent with the lack of a significant STING-independent IFN response in mouse fibroblasts, we found that HSPA8 phosphorylation did not occur in mouse cells. However, all primates tested, as well as rats, demonstrated intact HSPA8 phosphorylation, indicating that the SIDSP is broadly present in mammals and that laboratory mice specifically lost a robust SIDSP after their divergence from the common ancestor of mice and rats.

The cGAS-STING antiviral response has become the subject of intense development in the pharmaceutical industry, including efforts to develop inhibitors of cGAS and STING to treat human autoimmune diseases (33-35), as well as agonists of STING to improve immune responses to tumors (36-38). Our discovery of a second DNA-activated antiviral response in human cells has important implications for these efforts. Harnessing agonism of the DNA-PK SIDSP to trigger innate immune responses in the tumor microenvironment could broaden the toolkit of sophisticated adjuvant immunotherapies.

In summary, we have described the existence of a potent STING-independent DNA sensing pathway (SIDSP) in human cells, and we have identified its sensor, a unique target, two distinct viral antagonists, and a potent small molecule inhibitor of the response.

### Methods

### Reagents, Antibodies, and Inhibitors

Sheared CT DNA (Sigma) and 2′3′ cGAMP (Invivogen) were purchased and diluted in water; ISD oligos were ordered from Integrated DNA Technologies and annealed in water (30); RIG-I ligand was synthesized in vitro as previously described using HiScribe™ T7 High Yield RNA Synthesis Kit (39). For plasmid stimulations, midiprepped pcDNA3 was either untreated or sonicated with a Covaris M220 focused ultrasonicator at 5% ChIP (factory setting). Nu-7441 and Ku-60019 (SelleckChem) were suspended in DMSO and used to treat cells for 1 hour prior to stimulation with nucleic acid ligands. For Nu-7441, we used 0.25, 0.5, 1, or 2 μM. For Ku-60019, we used 0.125, 0.25, 0.5, or 1 μM. Untreated cells received the same amounts of plain DMSO.

### Cell Treatments

HEK 293 cells were grown in DMEM supplemented with 10% FCS, L-glutamine, penicillin/streptomycin, sodium pyruvate, and HEPES. U937 and THP1 cell lines were grown in RPMI supplemented as above, and differentiated prior to stimulation using 100 nM phorbol myristoyl acetate (PMA) for 24 hours and then rested in media lacking PMA for 24 hours.

HEK 293 cells were plated at 0.5 million/well in a 6 well dish in 2 mL media the day before stimulation for protein harvest. For RNA harvest and qPCR, U937 cells were plated at 0.25 million/well in a 24 well dish. In the 6 well dish format, cells received 8 μg of CT DNA, ISD100, or pcDNA3 complexed with 8 μl of Lipofectamine™ 2000. 10 μM cGAMP was complexed with 8 μl Lipofectamine™ and 1 μg RIG-I ligand was complexed with 1 μl Lipofectamine™ to achieve comparable induction of IFN across treatments in competent cells. Stimulations done in 24 well plates were scaled by ¼. Etoposide (prepared in DMSO) was diluted in culture media to 50 μM, and untreated cells received the same volume of DMSO. Cells were irradiated with 30 Gy using a Rad Source RS 2000 X-irradiator.

### IFN Bioassay

Supernatants from stimulated cells were harvested 24 hours post-stimulation and used to stimulate a HeLa cell line stably expressing an ISRE-luciferase reporter as described previously (11).

### Western Blotting

Cells were harvested by trypsinization (U937 cells) or vigorous wash with PBS (HEK293 cells), pelleted, and lysed using either a 1% Triton-X-100 buffer (20 mM HEPES, 150 mM NaCl, 10% glycerol, 1 mM EDTA, Pierce phosphatase/protease inhibitors) or, for samples requiring measurement of DNA-PK protein levels, RIPA buffer (150 mM NaCl, 1% Triton-X-100, 0.5% sodium deoxycholate, 0.1% SDS, 50 mM Tris pH 8.0, Pierce phosphatase/protease inhibitors). Lysates were vortexed and incubated on ice for 15 minutes before clearing by centrifugation for 15 minutes. Proteins were separated on Bolt 4-12% Bis-Tris gels (ThermoFisher) in MES buffer for 30 minutes at 200 V and transferred to Immobilon-FL PVDF membrane (Sigma). Blots were blocked in 5% BSA/TBST for 30 minutes prior to incubation with primary. The pIRF3 S386 blots were incubated at 4° C. overnight and washed at least 30 minutes in TBST prior to secondary incubation to prevent background. In order to better resolve DNA-PK (470 kDa), lysates were run on 3-8% Tris-acetate gels (ThermoFisher) for 2 hours at 150 V and then transferred in 5% methanol for 3 hours at 20 V at 4° C.

### LentiCRISPR Targeting

VSV-G pseudotyped, self-inactivating lentivirus was prepared by transfecting a 60-80% confluent 10-cm plate of HEK 293T cells with 1.5 μg of pVSV-G expression vector, 3 μg of pMDLg/pRRE, 3 μg pRSV-Rev and 6 μg of pRRL lentiCRISPR vectors using Poly(ethyleneimine) (PEI; Sigma). Media was replaced 24 hours post-transfection and harvested 24 hours later for filtration with a 0.45 μm filter (SteriFlip, Millipore). Approximately 1 million cells were transduced with 10 mL filtered virus. Targeting NHEJ components efficiently was difficult; best results were achieved by increasing transduction rates with sequential transductions on two consecutive days. Cells were plated for stimulations while still under selection at day 4 post first transduction.

For CRISPR/Cas9 gene targeting, we generated pRRL lentiviral vectors in which a U6 promoter drives expression of a gRNA, and an MND promoter drives expression of Cas9, a T2A peptide, and either a puromycin or blasticidin (40). gRNA sequences are as follows, where the (G) denotes a nucleotide added to enable robust transcription off the U6 promoter and the underlined sequence denotes the Protospacer Adjacent Motif (PAM): H1 off-target control: (G)ACGGAGGCTAAGCGTCGCAA (SEQ ID NO:1) (41); TMEM173 (STING): GGTGCCTGATAACCTGAGTATGG (SEQ ID NO:2) (40); TBK1: (G)CATAAGCTTCCTTCGTCCAGTGG (SEQ ID NO:3) (7); PRKDC (DNA-PK): GCAGGAGACCTTGTCCGCTGCGG (SEQ ID NO:4); XRCC6 (Ku70): GATCCGTGGCCCATCATGTCTTGG (SEQ ID NO:5); XRCC5 (Ku80): GTTGTGCTGTGTATGGACGTGGG (SEQ ID NO:6); ATM guide 1: (G)CCAAGGCTATTCAGTGTGCGAGG (SEQ ID NO:7) (41); ATM guide 2: (G)TGATAGAGCTACAGAACGAAAGG (SEQ ID NO:8) (41); and E1A (G)AAGACCTGCAACCGTGCCCGGGG (SEQ ID NO:9) (Lau, et al 2015) (11). Guides against PRKDC, XRCC6, and XRCC5 were designed using Benchling.

### Generation of Clonal Cell Lines

KO cell lines were generated by limiting dilution, screened by western blot, and verified by Sanger sequencing and functional assays. The STING/DNA-PK DKO U937 cell line was produced by transducing U937s simultaneously with a STING lentiCRISPR puro virus and a DNA-PK lentiCRISPR blasticidin virus, selecting in 10 μg/ml puro and 5 μg/ml blasticidin, and seeding in 96 well plates immediately after selection. Very few colonies grew, and the verified DKO clone grew markedly slower than H1 non-targeted control clones or the STING KO clones, as expected (19).

PCR primers used for amplifying genomic DNA surrounding CRISPR targeting sites in clonal lines were as follows (Forward/Reverse):

TMEM173:
(SEQ ID NO: 10)
5′-AGCTCCAGGCCCGGATTCG-3′
(SEQ ID NO: 11)
/5′-TGCCCGTTCTCCAGAAGCTC-3′
TBK1:
(SEQ ID NO: 12)
5′-CCCTACTGTATCCTCATG-3′
(SEQ ID NO: 13)
/5′-CTTACCTCCTCTTCAATAGC-3′
PRKDC:
(SEQ ID NO: 14)
5′-GGGGCATTTCCGGGTCCGGG-3′
(SEQ ID NO: 15)
/5′-TGCCCTGCCCCCCACTCTGC-3′

Amplicons were cloned using the Zero Blunt™ TOPO PCR Cloning kit (ThermoFisher), prepared as plasmids, and then several individual plasmids were sequenced. Sequencing alignments were made using Benchling™.

RNA Isolation and qPCR

Cells were harvested in Trizol before purification via Direct-zol™ RNA miniprep (Genesee Scientific) per manufacturer's instructions with an additional dry spin after disposing of the final wash to prevent carryover. cDNA was generated using EcoDry™ double primed premix (Clontech). qPCR was performed using iTaq supermix on the Bio-Rad CFX96 Real-Time system. Human gene PCR primer sequences are as follows: GAPDH Fwd: 5′-AACAGCCTCAAGATCATCAGC-3′ (SEQ ID NO:16), GAPDH Rev: 5′-CACCACCTTCTTGATGTCATC-3′ (SEQ ID NO:17) IFNB1 Fwd: 5′-ACGCCGCATTGACCATCTATG-3′ (SEQ ID NO:18), IFNB1 Rev: 5′-CGGAGGTAACCTGTAAGTCTGT-3′ (SEQ ID NO:19). Mouse primer sequences are as follows:

Hprt Fwd:
(SEQ ID NO: 20)
5′-GTTGGATACAGGCCAGATTTGTTG-3′,
Hprt Rev:
(SEQ ID NO: 21)
5′-GAGGGTAGGCTGGCCTATAGGCT-3′
Ifnb Fwd:
(SEQ ID NO: 22)
5′-GCACTGGGTGGAATGAGACTATTG-3′
Ifnb Rev:
(SEQ ID NO: 23)
5′-TTCTGAGGCATCAACTGACAGGTC-3′.

cGAMP Quantitation Assay

Cells were plated at 100,000 cells/well in a 24 well tissue culture dish. 24 hours later, cells were transfected with either 10 μg/ml CT DNA in Lipofectamine™ 2000 (Invitrogen; ratio of 1 μL Lipofectamine™ per 1 μg CT DNA; (32), or with an identical volume of Lipofectamine™ 2000 alone. 4 hours later, cells were harvested and lysates were prepared using cGAMP EIA assay protocol provided by manufacturer (Arbor Assays), in a volume of 200 μL sample suspension buffer.

mRNA-Seq and Analysis

Total RNA was added directly to lysis buffer from the SMART-Seq v4 Ultra Low Input RNA Kit for Sequencing (Takara), and reverse transcription was performed followed by PCR amplification to generate full length amplified cDNA. Sequencing libraries were constructed using the NexteraXT™ DNA sample preparation kit (IIlumina) to generate Illumina-compatible barcoded libraries. Libraries were pooled and quantified using a Qubit® Fluorometer (Life Technologies). Dual-index, single-read sequencing of pooled libraries was carried out on a HiSeq2500 sequencer (Illumina) with 58-base reads, using HiSeq™ v4 Cluster and SBS kits (IIlumina) with a target depth of 5 million reads per sample. Base calls were processed to FASTQs on BaseSpace™ (Illumina), and a base call quality trimming step was applied to remove low-confidence base calls from the ends of reads. The FASTQs were aligned to the human reference genome using the STAR aligner, and gene counts were generated using htseq-count. QC and metrics analyses were performed using the Picard family of tools (v1.134).

Exploratory analysis and statistics were run using R (version 3.5.1) and bioconductor (version 3.7). The gene count matrix was filtered by a row mean of ten or greater counts and normalized with EDGER. Log CPM transformation was performed using voom through the limma bioconductor package (3.34.8). Statistical analysis (including differential expression) was performed using the limma package (42, 43).

Co-expression was performed on genes statistically significant in the differential expression analysis (threshold: linear fold change >=|1.5| and FDR <=0.05) in at least one comparison. The union of these DE genes were loaded into R and filtered by known interferon signaling genes using all of the GO terms. Correlations (ward.2 clustering and euclidean distance) were run on the union of log 2FC using the WGCNA and heatmap.2 bioconductor packages in R (42, 44, 45).

### Immunoprecipitation and Mass Spectrometry

To identify HSPA8 using mass spectrometry, we performed immunoprecipitation of CT-DNA stimulated HEK293 cells using the antibody to IRF3 pS386 crosslinked to Dynabeads™ (ThermoFisher) overnight at 4 C, then washed three times in lysis buffer and two times in ammonium biocarbonate (50 mM) before peptide digestion (V5280, Promega). Peptides were loaded onto a 3-cm self-packed C18 capillary pre-column (Reprosil™ 5 μM, Dr. Maisch). After a 10-min rinse (0.1% Formic Acid), the pre-column was connected to a 25-cm self-packed C18 (Reliasil™ 3 μM, Orochem) analytical capillary column (inner diameter, 50-μm; outer diameter, 360-μm) with an integrated electrospray tip (˜1-μm orifice). Online peptide separation followed by mass spectrometric analyses was performed on a 2D-nanoLC system (nanoAcquity™ UPLC system, Waters Corp.). Peptides were eluted using a 150-min gradient with solvent A (H₂O/Formic Acid, 99.9:1 (v/v)) and B (Acetonitrile/Formic Acid, 99.9:1 (v/v)): 10 min from 0% to 10% B, 105 min from 10% to 40% B, 15 min from 40% to 80% B, and 20 minutes with 100% A. Eluted peptides were directly electrosprayed into a Orbitrap QExactive™ mass spectrometer (Thermo Fisher Scientific) equipped with a high energy collision cell (HCD). The mass spectrometer was operated in a data-dependent mode to automatically switch between MS and MS/MS acquisitions. Each full scan (from m/z 300-1500) was acquired in the Orbitrap™ analyzer (resolution=70,000), followed by MS/MS analyses on the top twenty most intense precursor ions that had charge states greater than one. The HCD MS/MS scans were acquired using the Orbitrap™ system (resolution=17,500) at normalized collision energy of 28%. The precursor isolation width was set at 2 m/z for each MS/MS scan and the maximum ion accumulation times were as follows: MS (100 ms), MS/MS (100 ms). MS/MS data files were searched using the Comet algorithm (46), and the data were further processed using the Institute for System's Biology's Trans-Proteomic Pipeline (47). Static modification of cysteine (carbamidomethylation; 57.02 Da) was used in the search.

### Cloning of HSPA8

PCR and InFusion™ cloning (Clonetech) were used to generate N-terminal HA-tagged WT and alanine mutant human HSPA8 constructs from HEK 293 cell cDNA. A murine HSPA8 cDNA clone (Transomic technologies, Clone ID BC089322) was used as template to generate the epitope-tagged mouse versions.

### ICP0 Expression and HSV-1 Infections

0.25 million STING KO HEK293s were seeded in 12 well format the day before transfection with 0, 1, 2 or 4 μg of ICP0 expression plasmid using Lipofectamine™ 2000 at a 1 μl:1 μg DNA ratio. Empty pcDNA3 was used to bring the total amount of transfected DNA up to 4 μg total. 24 hours post-transfection, the cells were treated with 4 μg CT DNA or 4 μL Lipofectamine™ 2000 alone and harvested 3 hours later in RIPA buffer with phosphatase inhibitors for analysis by western blot. Wild-type HSV-1 strain KOS and ICP0-null HSV-1 strain 7134 were prepared in Vero cells and ICP0-complemented Vero cells, respectively (52), using a MOI of 0.01 for 48 hrs before virus-containing media was collected, spun down to remove any cells, and aliquoted for storage at −80 C. Tittering was performed by serial dilution and plaque assay on the appropriate Vero cell line. Plaques were visualized by fixing/staining in 20% methanol with 0.2% crystal violet.

### EXPERIMENTAL REPLICATES AND STATISTICS

All experiments presented in this study, except the mRNA-Seq studies, were done two or more times, with biological triplicates for each condition in RT-qPCR experiments. Quantitative data were visualized and analyzed using GraphPad™ Prism software. Multiple unpaired t-tests with significance determined by Holm-Sidak method were used to compare differences between groups, unless otherwise noted for specific tests in figure legends. Significance is indicated as follows: *p<0.05. **p<0.01, ***p<0.001, ****p<0.0001.
1. D. Goubau, S. Deddouche, E. S. C. Reis, Cytosolic sensing of viruses. Immunity 38, 855-869 (2013).
2. J. T. Crowl, E. E. Gray, K. Pestal, H. E. Volkman, D. B. Stetson, Intracellular Nucleic Acid Detection in Autoimmunity. Annu Rev Immunol, (2017).
3. T. Li, Z. J. Chen, The cGAS-cGAMP-STING pathway connects DNA damage to inflammation, senescence, and cancer. J Exp Med 215, 1287-1299 (2018).
4. L. Sun, J. Wu, F. Du, X. Chen, Z. J. Chen, Cyclic GMP-AMP synthase is a cytosolic DNA sensor that activates the type I interferon pathway. Science 339, 786-791 (2013).
5. J. Wu et al., Cyclic GMP-AMP is an endogenous second messenger in innate immune signaling by cytosolic DNA. Science 339, 826-830 (2013).
6. H. Ishikawa, G. N. Barber, STING is an endoplasmic reticulum adaptor that facilitates innate immune signalling. Nature 455, 674-678 (2008).
7 S. Liu et al., Phosphorylation of innate immune adaptor proteins MAVS, STING, and TRIF induces IRF3 activation. Science 347, aaa2630 (2015).
8. G. N. Barber, STING: infection, inflammation and cancer. Nat Rev Immunol 15, 760-770 (2015).
9. Q. Chen, L. Sun, Z. J. Chen, Regulation and function of the cGAS-STING pathway of cytosolic DNA sensing. Nat Immunol 17, 1142-1149 (2016).
10. L. Unterholzner, The interferon response to intracellular DNA: why so many receptors? Immunobiology 218, 1312-1321 (2013).
11. L. Lau, E. E. Gray, R. L. Brunette, D. B. Stetson, DNA tumor virus oncogenes antagonize the cGAS-STING DNA-sensing pathway. Science 350, 568-571 (2015).
12. K. Honda, A. Takaoka, T. Taniguchi, Type I interferon [corrected] gene induction by the interferon regulatory factor family of transcription factors. Immunity 25, 349-360 (2006).
13. J. T. Crowl, D. B. Stetson, SUMO2 and SUMO3 redundantly prevent a noncanonical type I interferon response. Proc Natl Acad Sci USA 115, 6798-6803 (2018).
14. F. Civril et al., Structural mechanism of cytosolic DNA sensing by cGAS. Nature 498, 332-337 (2013).
15. X. Li et al., Cyclic GMP-AMP synthase is activated by double-stranded DNA-induced oligomerization. Immunity 39, 1019-1031 (2013).
16. A. N. Blackford, S. P. Jackson, ATM, ATR, and DNA-PK: The Trinity at the Heart of the DNA Damage Response. Mol Cell 66, 801-817 (2017).
17. S. E. Golding et al., Improved ATM kinase inhibitor KU-60019 radiosensitizers glioma cells, compromises insulin, AKT and ERK prosurvival signaling, and inhibits migration and invasion. Mol Cancer Ther 8, 2894-2902 (2009).
18. J. J. Leahy et al., Identification of a highly potent and selective DNA-dependent protein kinase (DNA-PK) inhibitor (NU7441) by screening of chromenone libraries. Bioorg Med Chem Lett 14, 6083-6087 (2004).
19. B. L. Ruis, K. R. Fattah, E. A. Hendrickson, The catalytic subunit of DNA-dependent protein kinase regulates proliferation, telomere length, and genomic stability in human somatic cells. Mol Cell Blot 28, 6182-6195 (2008).
20. G. Li, C. Nelsen, E. A. Hendrickson, Ku86 is essential in human somatic cells. Proc Natl Acad Sci USA 99, 832-837 (2002).
21. F. J. Fattah, N. F. Lichter, K. R. Fattah, S. Oh, E. A. Hendrickson, Ku70, an essential gene, modulates the frequency of rAAV-mediated gene targeting in human somatic cells. Proc Natl Acad Sci USA 105, 8703-8708 (2008).
22. Y. Gu, S. Jin, Y. Gao, D. T. Weaver, F. W. Alt, Ku70-deficient embryonic stem cells have increased ionizing radiosensitivity, defective DNA end-binding activity, and inability to support V(D)J recombination. Proc Natl Acad Sci USA 94, 8076-8081 (1997).
23. S. Lindquist, E. A. Craig, The heat-shock proteins. Annu Rev Genet 22, 631-677 (1988).
24. J. A. Neal, Y. Xu, M. Abe, E. Hendrickson, K. Meek, Restoration of ATM Expression in DNA-PKcs-Deficient Cells Inhibits Signal End Joining. J Immunol 196, 3032-3042 (2016).
25. J. Parkinson, S. P. Lees-Miller, R. D. Everett, Herpes simplex virus type 1 immediate-early protein vmw110 induces the proteasome-dependent degradation of the catalytic subunit of DNA-dependent protein kinase. J Virol 73, 650-657 (1999).
26. F. Stricher, C. Macri, M. Ruff, S. Muller, HSPA8/HSC70 chaperone protein: structure, function, and chemical targeting. Autophagy 9, 1937-1954 (2013).
27. B. J. Ferguson, D. S. Mansur, N. E. Peters, H. Ren, G. L. Smith, DNA-PK is a DNA sensor for IRF-3-dependent innate immunity. Elife 1, e00047 (2012).
28. N. E. Peters et al., A mechanism for the inhibition of DNA-PK-mediated DNA sensing by a virus. PLoS Pathog 9, e1003649 (2013).
29. S. R. Scutts et al., DNA-PK Is Targeted by Multiple Vaccinia Virus Proteins to Inhibit DNA Sensing. Cell Rep 25, 1953-1965 e1954 (2018).
30. D. B. Stetson, R. Medzhitov, Recognition of cytosolic DNA activates an IRF3-dependent innate immune response. Immunity 24, 93-103 (2006).
31. A. Y. Karpova, M. Trost, J. M. Murray, L. C. Cantley, P. M. Howley, Interferon regulatory factor-3 is an in vivo target of DNA-PK. Proc Natl Acad Sci USA 99, 2818-2823 (2002).
32. A. I. Walker, T. Hunt, R. J. Jackson, C. W. Anderson, Double-stranded DNA induces the phosphorylation of several proteins including the 90 000 mol. wt. heat-shock protein in animal cell extracts. EMBO J 4, 139-145 (1985).
33. J. Vincent et al., Small molecule inhibition of cGAS reduces interferon expression in primary macrophages from autoimmune mice. Nat Commun 8, 750 (2017).
34. J. Hall et al., Discovery of PF-06928215 as a high affinity inhibitor of cGAS enabled by a novel fluorescence polarization assay. PLoS One 12, e0184843 (2017).
35. S. M. Haag et al., Targeting STING with covalent small-molecule inhibitors. Nature 559, 269-273 (2018).
36. L. Corrales, S. M. McWhirter, T. W. Dubensky, Jr., T. F. Gajewski, The host STING pathway at the interface of cancer and immunity. J Clin Invest 126, 2404-2411 (2016).
37. K. E. Sivick et al., Magnitude of Therapeutic STING Activation Determines CD8(+) T Cell-Mediated Anti-tumor Immunity. Cell Rep 25, 3074-3085 e3075 (2018).
38. J. M. Ramanjulu et al., Design of amidobenzimidazole STING receptor agonists with systemic activity. Nature 564, 439-443 (2018).
39. T. Saito, D. M. Owen, F. Jiang, J. Marcotrigiano, M. Gale, Jr., Innate immunity induced by composition-dependent RIG-I recognition of hepatitis C virus RNA. Nature 454, 523-527 (2008).
40. E. E. Gray et al., The AIM2-like Receptors Are Dispensable for the Interferon Response to Intracellular DNA. Immunity 45, 255-266 (2016).
41. N. E. Sanjana, O. Shalem, F. Zhang, Improved vectors and genome-wide libraries for CRISPR screening. Nat Methods 11, 783-784 (2014).
42. G. K. Smyth, Linear models and empirical bayes methods for assessing differential expression in microarray experiments. Stat Appl Genet Mol Biol 3, Article3 (2004).
43. C. W. Law, Y. Chen, W. Shi, G. K. Smyth, voom: Precision weights unlock linear model analysis tools for RNA-seq read counts. Genome Biol 15, R29 (2014).
44. A. E. Loraine et al., Analysis and visualization of RNA-Seq expression data using RStudio, Bioconductor, and Integrated Genome Browser. Methods Mol Biol 1284, 481-501 (2015).
45. R. C. Gentleman et al., Bioconductor: open software development for computational biology and bioinformatics. Genome Biol 5, R80 (2004).
46. J. K. Eng, T. A. Jahan, M. R. Hoopmann, Comet: an open-source MS/MS sequence database search tool. Proteomics 13, 22-24 (2013).
47. A. Keller, J. Eng, N. Zhang, X. J. Li, R. Aebersold, A uniform proteomics MS/MS analysis platform utilizing open XML file formats. Mol Syst Biol 1, 2005 0017 (2005).

1. A method for treating of an autoimmune disease or an autoinflammatory disease, comprising administering to a subject in need thereof an amount effective of a DNA-dependent protein kinase (DNA-PK) inhibitor and/or an inhibitor of HSPA8/HSC70, to treat the autoimmune disorder or the auto-inflammatory disorder.

2. The method of claim 1, wherein the DNA-PK inhibitor and/or the HSPA8/HSC70 inhibitor are not inhibitors expressed by non-recombinant viruses.

3. The method of claim 1, wherein the method comprises administering the DNA-PK inhibitor to the subject, wherein the DNA-PK inhibitor comprises one or more of small molecule inhibitors of activity (such as kinase activity), antisense oligonucleotides directed against the DNA-PK DNA or mRNA; small interfering RNAs (siRNAs), short hairpin RNAs (shRNAs), microRNAs (miRNA) or small internally segmented interfering RNAs (sisiRNA) directed against the DNA-PK protein, DNA, or mRNA; DNA-PK antibodies, and aptamers that bind to DNA-PK.

4. The method of claim 1, wherein the DNA-PK inhibitor is a small molecule inhibitor.

5. The method of claim 4, wherein the DNA-PK small molecule inhibitor comprises one or more of NU-7441, M3814, Compound II (2-(Morpholin-4-yl)-benzo[h]chromen-4-one), or Compound III (1-(2-hydroxy-4-morpholinophenyl)ethan-1-one), or pharmaceutically acceptable salts, esters, or prodrugs thereof.

6. The method of claim 1, wherein the method comprises administering the HSPA8/HSC70 inhibitor to the subject.

7. The method of claim 6, wherein the HSPA8/HSC70 inhibitor comprises a small molecule inhibitor of activity, antisense oligonucleotides directed against the HSPA8/HSC70 DNA or mRNA; small interfering RNAs (siRNAs), short hairpin RNAs (shRNAs), microRNAs (miRNA) or small internally segmented interfering RNAs (sisiRNA) directed against the HSPA8/HSC70 protein, DNA, or mRNA; HSPA8/HSC70 antibodies, aptamers that bind to HSPA8/HSC70, and any other chemical or biological compound that can interfere with HSPA8/HSC70 expression, activity, and/or stability.

8. The method of claim 1, wherein the method further comprises administering an inhibitor of Cyclic GMP-AMP synthase (cGAS) expression, activity, and/or stability, and/or an inhibitor of Stimulator of interferon genes (STING), also known as transmembrane protein 173 (TMEM173)) expression, activity, and/or stability.

9. The method of claim 8, wherein the cGAS and/or STING inhibitor may include, but it not limited to, small molecule inhibitors, antisense oligonucleotides directed against the cGAS or STING DNA or mRNA; small interfering RNAs (siRNAs), short hairpin RNAs (shRNAs), microRNAs (miRNA) or small internally segmented interfering RNAs (sisiRNA) directed against the cGAS or STING protein, DNA, or mRNA; cGAS or STING antibodies, aptamers that bind to cGAS or STING, any other chemical or biological compound that can interfere with cGAS or STING expression, activity, and/or stability, PF-06928215, RU.521, and/or one or more STING inhibitors and/or cGAs inhibitors selected from the group consisting of:
STING inhibitors:
C-170 (N-(4-butylphenyl)-5-nitrofuran-2-carboxamide): 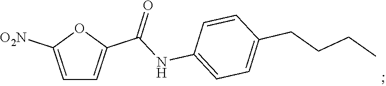 [SMILES: CCCCc1ccc(NC(=O)c2ccc([N+](=O)[O-])o2)cc1]
C-171 (N-(4-hexylphenyl)-5-nitrofuran-2-carboxamide): 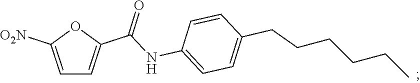 [SMILES: CCCCCCc1ccc(NC(=O)c2ccc([N+](=O)[O-])o2)cc1]
H-151 (1-(4-ethylphenyl)-3-(1H-indol-3-yl)urea): 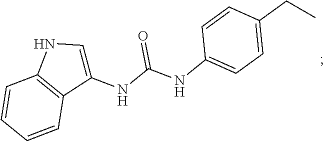 [SMILES: CCc1ccc(NC(=O)Nc2c[nH]c3ccccc23)cc1]
and
H-151-AL (1-(4-ethynylphenyl)-3-(1H-indol-3-yl)urea): 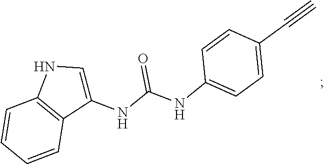 [SMILES: C#Cc1ccc(NC(=O)Nc2c[nH]c3ccccc23)cc1]
cGAS inhibitors:
5-phenyltetrazolo[1,5-a]pyrimidin-7-ol (compound 15): 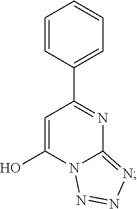 [SMILES: Oc1cc(-c2ccccc2)nc2nnnn12]
7-hydroxy-N-methyl-5-phenylpyrazolo[1,5-a]pyrimidine-3-carboxamide (compound 16): 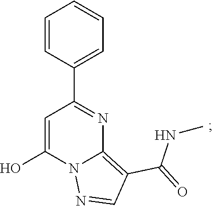 [SMILES: CNC(=O)c1cnn2c(O)cc(-c3ccccc3)nc12]
7-hydroxy-N-(2-hydroxyethyl)-5-phenylpyrazolo[1,5-a]pyrimidine-3-carboxamide (compound 17): 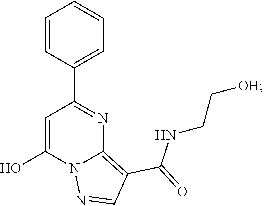 [SMILES: O=C(NCCO)c1cnn2c(O)cc(-c3ccccc3)nc12]
(7-hydroxy-5-phenylpyrazolo[1,5-a]pyrimidine-3-carbonyl)glycine (compound 18): 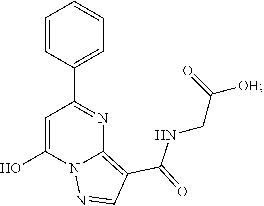 [SMILES: O=C(O)CNC(=O)c1cnn2c(O)cc(-c3ccccc3)nc12]
and
(S)-7-hydroxy-N-(1-hydroxypropan-2-yl)-5-phenylpyrazolo[1,5-a]pyrimidine-3-carboxamide (compound 19): 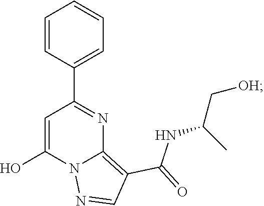 [SMILES: C[C@@H](CO)NC(=O)c1cnn2c(O)cc(-c3ccccc3)nc12]
or pharmaceutically acceptable salts, esters, or prodrugs thereof.

10. The method of claim 1, wherein the subject has an autoimmune disease.

11. The method of claim 10, wherein the autoimmune disease comprises one or more of Systemic lupus erythematosus (SLE), Discoid lupus, Cutaneous lupus, Sjogrens syndrome, Aicardi-Goutieres syndrome (AGS), pemphigoid (any type), Crohn's disease, endometriosis, fibromyalgia, glomerulonephritis, juvenile arthritis, type 1 diabetes, multiple sclerosis, psoriasis, rheumatoid arthritis, sarcoidosis, scleroderma, and ulcerative colitis.

12. The method of claim 10, wherein the autoimmune disease comprises one or more of Systemic lupus erythematosus (SLE), Discoid lupus, Cutaneous lupus, Sjogrens syndrome, and Aicardi-Goutieres syndrome (AGS).

13. The method of claim 10, wherein the autoimmune disease comprises Cutaneous lupus.

14. The method of claim 10, wherein the autoimmune disease comprises scleroderma.

15. The method of claim 1, wherein the subject has an autoinflammatory disease.

16. A method for monitoring therapy of a subject being treated for an autoimmune disease and/or an autoinflammatory disease, comprising
(a) determining a baseline level of HSPA8/HSC70 phosphorylation in a biological sample from the subject; and
(b) determining level of HSPA8/HSC70 phosphorylation in a biological sample from the subject 1 or more (2, 3, 4, 5, 6, or more times) after treatment for the autoimmune disease and/or an autoinflammatory disease,
wherein a decrease in HSPA8/HSC70 phosphorylation in the biological sample from the subject after treatment indicates efficacy of the therapy, and wherein an increase in HSPA8/HSC70 phosphorylation in the biological sample from the subject after treatment indicates that the therapy was ineffective.

17.-21. (canceled)

22. A pharmaceutical composition comprising:
(a) a DNA-PK inhibitor and/or an inhibitor of HSPA8/HSC70; and
(b) an inhibitor of cGAS expression, activity, and/or stability, and/or an inhibitor of STING expression, activity, and/or stability.

23. The pharmaceutical composition of claim 22, wherein the DNA-PK inhibitor and/or an inhibitor of HSPA8/HSC70 comprises a DNA-PK inhibitor.

24. The pharmaceutical composition of claim 23, wherein the DNA-PK inhibitor comprises one or more of small molecule inhibitors of activity (such as kinase activity), antisense oligonucleotides directed against the DNA-PK DNA or mRNA; small interfering RNAs (siRNAs), short hairpin RNAs (shRNAs), microRNAs (miRNA) or small internally segmented interfering RNAs (sisiRNA) directed against the DNA-PK protein, DNA, or mRNA; DNA-PK antibodies, and aptamers that bind to DNA-PK.

25.-35. (canceled)
